(19) United States
(12) Patent Application Publication
(10) Pub. No.: US 2021/0371901 A1
(43) Pub. Date: Dec. 2, 2021

(54) Kits and Methods for Detecting Methylated DNA

(71) Applicant: Sequenom, Inc., San Diego, CA (US)

(72) Inventor: Michael Rehli, Regensburg (DE)

(73) Assignee: Sequenom, Inc., San Diego, CA (US)

(21) Appl. No.: 17/385,657

(22) Filed: Jul. 26, 2021

Related U.S. Application Data
(63) Continuation-in-part of application No. 16/597,877, filed on Oct. 10, 2019.
Application No. 16/597,877 is a continuation of application No. 14/996,882, filed on Jan. 15, 2016, now Pat. No. 10,487,351.
Application No. 14/996,882 is a continuation of application No. 11/720,300, filed on Aug. 16, 2007, now Pat. No. 9,249,464.
Application No. 11/720,300 is a continuation-in-part of application No. 17/121,923, filed on Dec. 15, 2020.
Application No. 17/121,923 is a continuation of application No. 15/876,844, filed on Jan. 22, 2018, now abandoned.
Application No. 15/876,844 is a continuation of application No. 15/679,861, filed on Aug. 17, 2017, now abandoned.
Application No. 15/679,861 is a continuation of application No. 14/734,369, filed on Jun. 9, 2015, now Pat. No. 9,873,919.
Application No. 14/734,369 is a continuation of application No. 11/569,051, filed on Nov. 13, 2006, now Pat. No. 9,074,013.

(30) Foreign Application Priority Data
Nov. 29, 2004 (EP) 04 02 8268.3

(51) Int. Cl. C12Q 1/6804; C12Q 1/6886

(52) U.S. Cl. CPC C12Q 1/6804; C12Q 2600/16; C12Q 2600/154; C12Q 1/6886

(57) ABSTRACT

The present invention relates to an in vitro method for detecting methylated DNA comprising (a) coating a container with a polypeptide capable of binding methylated DNA; (b) contacting said polypeptide with a sample comprising methylated and/or unmethylated DNA; and (c) detecting the binding of said polypeptide to methylated DNA. In a preferred embodiment, said method further comprises step (d) analyzing the detected methylated DNA by sequencing. Another aspect of the present invention is a kit for detecting methylated DNA according to the methods of the invention comprising (a) a polypeptide capable of binding methylated DNA; (b) a container which can be coated with said polypeptide; (c) means for coating said container; and (d) means for detecting methylated DNA.

### RELATED PATENT APPLICATIONS

This application is a continuation-in-part of U.S. patent application Ser. No. 16/597,877, filed on Oct. 10, 2019, which is a continuation application of U.S. patent application Ser. No. 14/996,882, filed Jan. 15, 2016, issued as U.S. Pat. No. 10,487,351. Said application is a continuation of U.S. patent application Ser. No. 11/720,300 filed Aug. 16, 2007, issued as U.S. Pat. No. 9,249,464, entitled “KITS AND METHODS FOR DETECTING METHYLATED DNA”, naming Michael Rehli as inventor which is a national stage of International Patent Application PCT/EP2005/012705 filed on Nov. 28, 2005 entitled “KITS AND METHODS FOR DETECTING METHYLATED DNA”, naming Michael Rehli as applicant and inventor which claims the benefit of EP 04 02 8268.3 filed Nov. 29, 2004 entitled “KITS AND METHODS FOR DETECTING METHYLATED DNA” naming Michael Rehli as inventor. The entire content of these applications are incorporated herein by reference, including, without limitation, all text, tables, and drawings, for all purposes.

This application is also a continuation-in-part of U.S. application Ser. No. 17/121,923 filed on Dec. 15, 2020, which is a continuation of U.S. application Ser. No. 15/876,844, filed on Jan. 22, 2018, which is a continuation of U.S. application Ser. No. 15/679,861, filed on Aug. 17, 2017, which is a continuation of U.S. application Ser. No. 14/734,369, filed on Jun. 9, 2015, which is a continuation of U.S. patent application Ser. No. 11/569,051 filed Nov. 13, 2006, entitled “MEANS AND METHODS FOR DETECTING METHYLATED DNA”, naming Michael Rehli as inventor which is a national stage of International Patent Application PCT/EP2005/12707 filed on Nov. 28, 2005 entitled “MEANS AND METHODS FOR DETECTING METHYLATED DNA”, naming Michael Rehli as applicant and inventor which claims the benefit of EP 04 02 8267.5 filed Nov. 29, 2004 entitled “MEANS AND METHODS FOR DETECTING METHYLATED DNA”, naming Michael Rehli as inventor. The entire content of the aforementioned patent applications are incorporated herein by reference, including, without limitation, all text, tables, and drawings, for all purposes.

### OVERVIEW

The present invention relates to an in vitro method for detecting methylated DNA comprising (a) coating a container with a polypeptide capable of binding methylated DNA; (b) contacting said polypeptide with a sample comprising methylated and/or unmethylated DNA; and (c) detecting the binding of said polypeptide to methylated DNA. In a preferred embodiment, said method further comprises step (d) analyzing the detected methylated DNA by sequencing. Another aspect of the present invention is a kit for detecting methylated DNA according to the methods of the invention comprising (a) a polypeptide capable of binding methylated DNA; (b) a container which can be coated with said polypeptide; (c) means for coating said container; and (d) means for detecting methylated DNA.

The information to make the cells of all living organisms is contained in their DNA. DNA is made from 4 bases abbreviated as G, A, T, and C, and is built like a very long ladder with pairs of these letter making up each of the “rungs” of the ladder. The letter G pairs with C and A with T. Strings of these pairs store information like a coded message, with the information to make specific molecules grouped into regions called genes. Every cell of diploid animals contains two copies of every gene, with one copy of each gene coming from the mother and one copy from the father. (The only exceptions to this rule are genes on chromosomes that determine whether organisms develop as a “male” or a “female.”)

### DNA Methylation and Gene Regulation

Apart from the four bases—adenine, guanine, cytosine and thymine—that “spell” our genome, there also is a fifth base which is produced by the modification of the post-replicative DNA. DNA methyl transferases (DNMTs) can catalyse the transfer of a methyl group from the methyl donor S-adenosylmethionine to the cytosine ring, and thereby produce the base 5-methylcytosine. Specific cytosine residues are modified in mammals, which precede a guanosine residue in the DNA sequence (CpG dinucleotide) (Singal, Blood 93 (1999), 4059-4070); Robertson, Nat. Rev. Genet. 1 (2000), 11-19; Ng, Curr. Opin. Genet. Dev. (2000), 158-163; Razin, EMBO J. 17 (1998), 4905-4908). The methylation of CpG dinucleotides generally correlates with stable transcriptional repression and presumably leads to the fact that large parts of the non-coding genome and potentially harmful sequences such as transposons, repeats or viral inserts are not transcribed. It is interesting that CpG dinucleotides are very unevenly distributed in the genome (Singal (1999), loc. cit., Robertson (2000), loc. cit., Ng (2000), loc. cit., Razin (1998), loc. cit.). A large part of the genome contains much fewer CpGs than is statistically expected. This is presumably due to the fact that 5-methylcytosine deaminates comparatively easily to thymidine, which, in the course of evolution, leads to a relative decrease in the number of CpG dinucleotides. There are, however, again and again, larger numbers of CpGs distributed within the genome, so-called CpG islands. These regions often contain transcription initiation points and gene promoters and are generally not methylated in contrast to the CpGs which are not associated with CpG islands. In normal cells, the methylation of CpG islands has been observed only in exceptional cases such as the inactivation of the second copy of the X-chromosome in female cells and the parental imprinting genome (Singal (1999), loc. cit., Robertson (2000), loc. cit., Ng (2000), loc. cit., Razin (1998), loc. cit.).

### Regulation of DNA Methylation

It is only partly understood how DNA methylation patterns are established in the course of the embryogenesis and how the CpG methylation is maintained and regulated in the genome (Singal (1999), loc. cit., Ng (2000), loc. cit., Razin (1998), loc. cit.). In mammal species, there are three DNA methyl transferases known (DNMT1, 3a and 3b) which catalyse the DNA methylation process. The corresponding share that each DNMT contributes to the maintenance and regulation of the CpG methylation must, however, still be clarified. Yet, all three enzymes are obviously essential to embryogenesis, the corresponding knockout mice die in utero or shortly after birth (Bestor, Hum. Mol. Genet. 9 (2000), 2395-2402; El Osta, Bioessays 25 (2003), 1071-1084). In the meantime, the connection between DNA methylation, modifications of the chromatin structure and certain histone modifications has been shown several times. The methylation of DNA mostly correlates with histone deacetylation and methylation of the lysine 9 residue at histone H3 (Sims, Trends Genet. 19 (2003), 629-639, Fahrner, Cancer Res. 62 (2002), 7213-7218). Accordingly, DNMTs are associated with histone deacetylases (HDACs) or co-repressor complexes. It is also hardly known how methyl groups are removed from CpG residues. In proliferating cells, the DNA methylation can probably also take place passively during replication. There are, however, also examples of DNA demethylation in post-mitotic cells which can be explained by the existence of an active, yet unknown demethylase (Wolffe, Proc. Natl. Acad. Sci. 96 (1999), 5894-5896).

### CpG Methylation and Gene Silencing

Methylation of promoters (but not of non-regulating sequences) correlates with stable, transcriptional repression (Singal (1999), loc. cit., Ng (2000), loc. cit., Razin (1998), loc. cit.). The repressive properties of 5-methylcytosine can be mediated by two mechanisms. Firstly, the DNA methylation can directly impair the binding of transcription factors. The second possibility, which is likely to be responsible for the largest part of repression, is the recruitment of methyl-CpG-binding proteins (MBPs) (Ballestar, Eur. J. Biochem. 268 (2001), 1-6). MBPs such as MECP2 or MBD2 (a component of the MeCP1 complex) are accompanied by co-repressor complexes and HDACs which have a repressive effect and are responsible for the formation of dense chromatin structures inaccessible to transcription factors (heterochromatin) (Ballestar (2001), loc. cit.).

### Epigenetic Changes in Tumorigenesis

It keeps becoming clearer that the formation of tumors is supported not only by genetic lesions (e.g. mutations or translocations) but also by epigenetic changes. An abnormal chromatin structure or DNA methylation can influence the transcriptional status of oncogenes or tumor suppressor genes and can promote tumor growth. Changes in the DNA methylation include either the loss of methylation in normally methylated sequences (hypomethylation) or the methylation of normally unmethylated sequences (hypermethylation) (Roberston (2000), loc. cit., Herman, N. Engl. J. Med. 349 (2003), 2042-2054; Momparler, Oncogene 22 (2003), 6479-6483; Esteller, Science 297 (2002), 1807-1808; Plass, Hum. Mol. Genet 11 (2002), 2479-2488).

### Hypomethylation

A global DNA hypomethylation has been described for almost all kinds of tumors. In tumor tissue, the content in 5-methylcytosine is reduced compared to normal tissue with the major share of demethylation events being found in repetitive satellite sequences or in centromere regions of the chromosomes. However, in single cases, the demethylation and activation of proto-oncogenes such as, e.g., bcl-2 or c-myc have also been described (Costello, J. Med. Genet. 38 (2001), 285-303).

### Hypermethylation of CpG Islands

CpG islands in general exert gene regulatory functions. This is why a change in the status of methylation correlates mostly directly with a change in the transcriptional activity of the locus concerned (Robertson (1999); Herman (2003); Esteller (2002); Momparler (2003); Plass (2002), all loc. cit.). Most CpG islands are present in unmethylated form in normal cells. In certain situations, CpG islands can, however, also be methylated in gene regulatory events. The majority of CpG islands of the inactivated X-chromosome of a female cell are, for example, methylated (Goto, Microbiol. Mol. Biol. Rev. 62 (1998), 362-378). CpG islands can be methylated also in the course of normal aging processes (Issa, Clin. Immunol. 109 (2003), 103-108).

It is in particular in tumors that CpG islands which are normally not methylated can be present in a hypermethylated form. In many cases, genes affected by the hypermethylation encode proteins which counteract the growth of a tumor such as, e.g., tumor suppressor genes. The following Table 1 lists examples of genes for which it could be shown that they can be inactivated in tumors through the epigenetic mechanism of hypermethylation.

TABLE 1
Hypermethylated genes in tumors (examples)

| | gene | chromosome | function |
|---|---|---|---|
| cell cycle control | p16 | 9p21 | cycline-dependent kinase inhibitor |
| | p15 | 9p21 | cycline-dependent kinase inhibitor |
| | Rb | 13q14 | cell cycle inhibition |
| | p73 | 1p36 | p53-like protein |
| DNA repair | MLH1 | 3p21 | DNA mismatch repair protein |
| | GSTPI | 11q13 | inhibitor of oxidative DNA damage |
| | O6-MGMT | 10q26 | DNA methyltransferase |
| | BRCA1 | 17q21 | DNA repair protein |
| apoptosis | TMS-1/ASC | 16p12-p11 | adaptor for caspase 1 |
| | caspase 8 | 2q33-q34 | PCD initiator (Fas, Trail, TNF, . . . ) |
| | DAPK1 | 9q34 | PCD by IFNγ |
| invasion/architecture | E-cadherin | 16q22 | adhesion molecule |
| | VHL | 3p26-p25 | angiogenesis-promoting protein |
| | TIMP-3 | 22q12-q13 | metalloproteinase inhibitor |
| | THBS1 | 15q15 | angiogenesis inhibitor |
| growth factor response | ER-α | 6q25 | estrogen receptor |
| | RAR-β | 3p24 | retinoic acid receptor |
| | SOCS-1 | 16p13 | neg. regulator in the JAK/STAT signal path |

Reasons for the tumor-specific hypermethylation are almost unknown. Interestingly, certain kinds of tumors seem to have their own hypermethylation profiles. It could be shown in larger comparative studies that hypermethylation is not evenly distributed but that it occurs depending on the tumor. In cases of leukaemia, mostly other genes are hypermethylated compared to, for instance, colon carcinomas or gliomas. Thus, hypermethylation could be useful for classifying tumors (Esteller, Cancer Res. 61 (2001), 3225-3229; Costello, Nat. Genet. 24 (2000), 132-138).

In many cases, hypermethylation is also combined with an increased activity of HDACs. After treatment with demethylating substances (e.g. 5-azacytidine), many methylated genes could only be reactivated after also using HDAC inhibitors (such as, e.g., trichostatin A (TSA)) (Suzuki, Nat. Genet. 31 (2002), 141-149; Ghoshal, Mol. Cell. Biol. 22 (2002), 8302-8319; Kalebic, Ann. N.Y. Acad. Sci 983 (2003), 278-285).

Most analyzes suggest that the DNA methylation is dominantly repressed and that it cannot be reversed by a treatment with HDAC inhibitors such as TSA (Suzuki (2002); Ghoshal (2002), loc. cit.). There are, however, also more recent indications that valproate, a HDAC inhibitor which is already used in clinics, can lead to the demethylation of DNA (Detich, J. Biol. Chem. 278 (2003), 27586-27592). However, no systematic analyzes have so far been carried out in this respect.

### Clinical Approaches for Reversing Epigenetic Changes

While genetic causes of cancer (such as, e.g., mutations) are irreversible, epigenetic changes contributing their share to the tumorigenesis might possibly be reversible. Thus, the possible treatment of epigenetic changes offers new possibilities of therapy for the treatment of neoplasias (Herman (2003); Momparler (2003); Plass (2002), all loc. cit.; Leone, Clin. Immunol. 109 (2003), 89-102; Claus, Oncogene 22 (2003), 6489-6496).

More than 20 years ago, 5-azacytidine has already been developed as an anti-neoplastic medicament and used without the molecular effect of the substance being known. Nowadays, it is already used successfully in a further developed form (Deoxy-5-azacytidine, Decitabine) for the treatment of myelodysplastic syndromes and secondary leukaemia (Leone (2003), loc. cit.; Lyons, Curr. Opin. Investig. Drugs 4 (2003), 1442-1450; Issa, Curr. Opin. Oncol. 15 (2003), 446-451). Due to the in vitro observation that HDAC inhibitors can support the reactivation of methylated promoters and can act synergistically with demethylated substances, at present pilot studies are carried out throughout the world, combining the use of both classes of substances (Kalebic (2003); Claus (2003), loc. cit.; Gagnon, Anticancer Drugs 14 (2003), 193-202; Shaker, Leuk. Res. 27 (2003), 437-444).

### Detection Methods for the Analysis of CpG Methylation

The development of detection methods for the analysis of genomic CpG methylation has mainly gained importance due to the fact that it has been found that changes in the CpG methylation pattern can be associated with diseases such as cancer. At present, there are mainly techniques known which are used for the detection of the CpG methylation of known gene loci (Dahl, Biogerontology 4 (2003), 233-250). Methods allowing an analysis of the CpG methylation throughout the genome are less established. In the following, the most common methods for analysis of CpG methylation together with their main fields of application are summarised.

### Use of Methylation-Sensitive Restriction Enzymes for the Detection of CpG Methylation

The methylation status of specific CpG dinucleotides can be determined using isoschizomers of bacterial restriction endonucleases which are characterised by different sensitivities vis-à-vis 5-methylcytosine. Examples thereof are the enzymes HpaII and MspI—both cut CCGG sequences, HpaII however only if the internal cytosine is not methylated. Some assays are based on the use of methylation-sensitive restriction enzymes, said assays being used for both the analysis of individual genes and analysis of the CpG methylation throughout the genome. The fragments of a methylation-sensitive restriction digestion are mostly detected by means of Southern blot or a genomic PCR of the region flanking the restriction site(Dahl (2003), loc. cit.). All analyzes of the CpG methylation throughout the genome, which have been published up to today, use methylation-sensitive restriction enzymes as a component of the method. Restriction Landmark Genomic Scanning (RLGS) (Costello, Methods 27 (2002), 144-149), for instance, uses a kind of two-dimensional agarose gel electrophorese in which every dimension is digested with a different methylation-sensitive restriction enzyme to identify differences in the CpG methylation of two DNA populations. Methylated CpG Island Amplification (MCA) enriches fragments with methylated SmaI restriction sites and uses LM-PCR for enriching the fragments. Such amplification products have already been successfully analyzed by means of Representational Difference Analysis (RDA) (Smith, Genome Res. 13 (2003), 558-569) or CpG island microarrays (Yan, Cancer Res. 6 (2001), 8375-8380).

With regard to the analysis of the CpG methylation throughout the genome, all assays that are based on methylation-sensitive restriction enzymes have disadvantages. In order to carry out the assays in an optimal way, it has, amongst others, to be guaranteed that all restriction digestions are completed. The greatest disadvantage is that the analyzes merely inform on the methylation status of the cytosine residues which have been recognised by the methylation-sensitive restriction enzymes used. The selection of the restriction enzymes automatically limits the number of detectable sequences—a neutral analysis of the CpG methylation is therefore not possible.

### Bisulfate Treatment for the Analysis of the CpG Methylation

The treatment of double-stranded genomic DNA with sodium bisulfate leads to the deamination of unmethylated cytosine residues into uracil residues and to the formation of two single strands that are no longer complementary. During this treatment, 5-methylcytosine is maintained. The differences in sequence produced in this way form the basis of the differentiation between methylated and unmethylated DNA (Frommer, Proc. Natl. Acad. Sci. 889 (1992), 1827-1831). DNA treated with bisulfite can be used directly in PCR in which uracil residues (previously unmethylated cytosine) and thymidine residues are amplified as thymidine and only 5-methylcytosine residues are amplified as cytosine residues. Depending on the application, the primers used for the PCR differentiate between methylated and unmethylated sequences or amplify fragments independently of the methylation status. PCR fragments which have been amplified using non-discriminating primers can, for instance, be sequenced directly to determine the share in methylated and unmethylated CpGs. Further methods make use of the physical differences of such PCR fragments (melting behaviour, single-strand conformation, restriction sites for restriction enzymes, etc.) for determining the degree of methylation (Dahl (2003), loc. cit.). Other methodical approaches that allow high throughput methylation analyzes utilise the differences in sequence for the specific amplification of methylated and unmethylated sequences by discriminating primers or probes (methylation-specific PCR, methylight PCR) (Dahl (2003), loc. cit.). Bisulfate-induced differences in the sequence of PCR products can also be found by means of methylation-specific oligonucleotide (MSO) microarrays (Shi, J. Cell. Biochem. 88 (2003), 138-143; Adorjan, Nucleic Acid Res. 30 (2002), e21; Gitan, Genome Res. 12 (2002), 158-164).

In contrast to the methylation-sensitive restriction enzymes, the DNA treated with bisulfate can provide information on the methylation status of several CpG residues in an amplified genomic fragment. The detection of CpG methylation by using discriminating primers or probes, however, is limited to the methylation status of single (or few) cytosine residues. Hence, the information provided by all presently known assays of the prior art that are suitable for high throughput methylation analysis of single gene loci is limited to one or only a few CpG residues within the gene of interest.

### Further Methods for the Detection of CpG Methylation

Antibodies against 5-methyl cytosine recognise CpG methylation in denatured, single-stranded DNA are used mainly for the immunohistochemical staining of the CpG methylation on the chromosomes of individual, fixed cells.

Already in 1994, the laboratory of A. Bird developed a method for enriching methylated DNA fragments by means of affinity chromatography (Cross, Nat. Genet. 6 (1994), 236-244). A recombinant MECP2 bound to a matrix was used for binding the methylated DNA. Since then this technique has been used, improved and combined with further techniques by other working groups (Shiraishi, Proc. Natl. Acad. Sci. 96 (1999), 2913-2918; Brock, Nucleic Acid. Res. 29 (2001), E123). The binding of strongly or less strongly methylated genomic sequences to an affinity matrix depends on the salt concentration which makes it possible to separate the CpG islands with dense methylation from other sequences with a lower methylation density. The disadvantage of this affinity chromatography is the large amount of genomic DNA required (50-100 μg) and the relatively time-consuming procedure.

In view of the foregoing, it is evident that methylation of CpG dinucleotides is an important epigenetic mechanism for controlling transcriptional activity of a cell. Generally, methylation of CpG dinucleotides correlates with transcriptional inactivity. Yet, during normal or degenerated differentiation processes the methylation pattern of genloci may change. Accordingly, the reversal of normal methylation patterns during tumorigenesis can lead to an abnormal repression (or activation) of genes, for instance, tumor suppressor genes or oncogenes, respectively, and, thus, leading to tumorigenesis. Hence, the detection of CpG methylated DNA and thus the identification of misregulated tumor-suppressor genes and/or oncogenes is of outmost clinical interest. As mentioned above, the prior art describes different approaches for the detection of methylated DNA which, however, suffer from certain shortcomings. For example, the methods of the prior art may not be suitable for high-through put applications or may not reliable detect CpG methylated DNA, particularly if only low amounts of DNA can be made subject of an analysis. Thus, there is still a need for further means and methods for detecting methylated DNA which may overcome the shortcomings and drawbacks of the prior art. Accordingly, the technical problem underlying the present invention is to comply with the needs described above.

The solution to this technical problem is achieved by providing the embodiments characterized in the claims.

Accordingly, in a first aspect the present invention relates to an in vitro method for detecting methylated DNA comprising
(a) coating a container with a polypeptide capable of binding methylated DNA;
(b) contacting said polypeptide with a sample comprising methylated and/or unmethylated DNA; and
(c) detecting the binding of said polypeptide to methylated DNA.

As documented in the appended Examples, it was surprisingly found that a single-tube assay/in vitro method can be safely and reliable employed in the detection of methylated nucleic acid molecules, in particular CpG-methylated DNA molecules/DNA fragments. The advantages of said method are its fast, sensitive, and reliable detection of preferably methylated DNA and its ability to analyze target DNA fragments according to their methylation degree. In contrast to the prior art, the method provided herein does not require bisulfate treatment or methylation-sensitive restriction and is not limited to detecting single/few CpG residues. The information provided may actually be more relevant than that of other methods of the prior art, since, methylation density of a proximal promoter can correlate better with the transcriptional status of a gene than the methylation status of a single CpG residue within the region. Accordingly, a “single-tube” assay is provided herein, wherein the degree of methylation may be estimated relative to a PCR reaction of the (genomic) input DNA.

A preferably homogeneously coated container in accordance with this invention, preferably, facilitates that a polypeptide which is capable of binding methylated DNA and which is employed in accordance with the method described herein has a maximum binding capacity for methylated DNA. A homogenous coating of the container can be achieved by methods known in the art and preferably by the method of the present invention described herein and/or in the appended Examples. Further, homogeneous coating can be controlled by methods known in the art, such as Coomassie-Blue staining. The term “container” encompasses any container which is commonly used and/or suitable for scientific and/or diagnostic purposes. Preferably, said container is composed of the following materials: polystyrene, polyvinyl chloride or polypropylene or the like, more preferably it is composed of polycarbonate. It is also preferred that polystyrene, polyvinyl chloride, polypropylene or polycarbonate is thermocycler-compatible, i.e. it is preferably heat-stable and/or durable at different temperatures for different time intervals. It is moreover preferred that polystyrene, polyvinyl chloride, polypropylene or polycarbonate are inert to chemical and/or biological agents used in connection with the method of the present invention.

So far, coatable PCR-tubes have only been used for immuno-polymerase chain reaction (immuno-PCR) (Sano, Science 258 (1992); Adler, Biochem Biophys Res Commun. 308 (2003), 240-250). Immuno-PCR is an antigen detection system, in which a specific DNA molecule is used as the marker. A streptavidin-protein A chimera that possesses tight and specific binding affinity both for biotin and immunoglobulin G is used to attach a biotinylated DNA specifically to antigen-monoclonal antibody complexes that are immobilized on microtiter plate wells. Then, a segment of the attached DNA is then amplified by PCR. Immuno-PCR is comparable to traditional ELISA techniques and uses the sandwich-approach with a more sensitive detection system (PCR detection of the marker DNA). Thus, in contrast to the present invention, where DNA is the direct subject of detection, Immuno-PCR uses DNA as a means (marker) to detect an antigen.

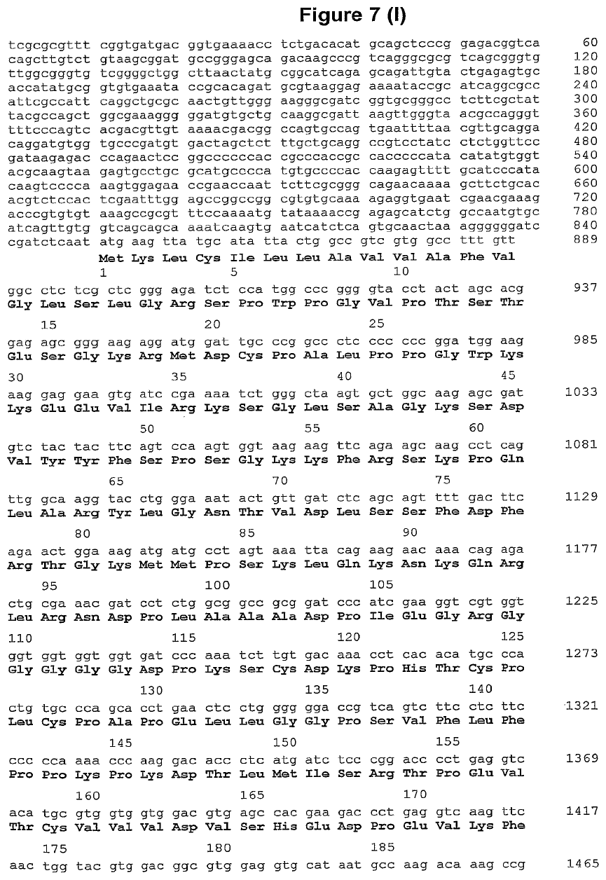

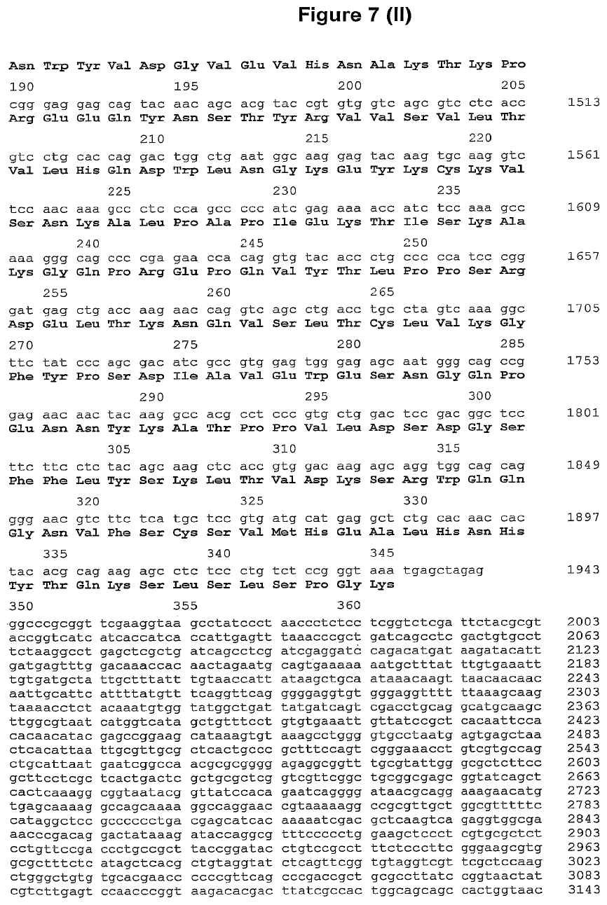

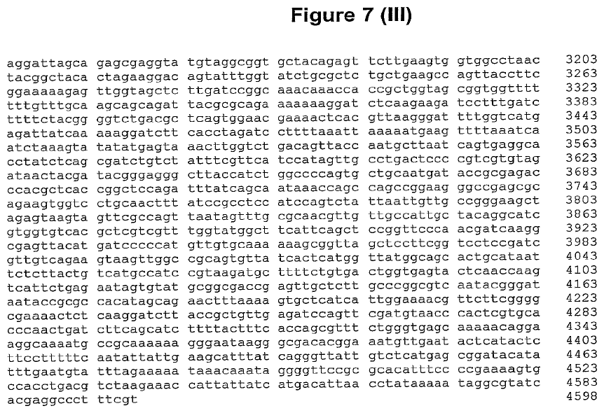

The term “coating” means that the surface of the container is preferably entirely coated with a polypeptide which is capable of binding methylated DNA, whereby essentially identical amounts of said polypeptide are present in each and every area of the surface of said container. Examples of such binding polypeptides are given herein below and comprise, inter alia and preferably, a polypeptide belonging to the Methyl-DNA binding protein (MBD) family, and most preferably a bifunctional polypeptide comprising the DNA-binding domain of a protein belonging to the family of Methyl-CpG binding proteins (MBDs) and an Fc portion of an antibody. Said DNA-binding domain is described herein below. Optionally, said bifunctional polypeptide comprises a polypeptide linker which is described herein below. Accordingly, said bifunctional polypeptide is preferably characterized by the amino acid sequence shown in SEQ ID NO: 2 (FIG. 7) which is encoded by the nucleotide sequence shown in SEQ ID NO: 1 (FIG. 7)

The term “polypeptide capable of binding methylated DNA” encompasses any polypeptide which can bind methylated DNA as described herein. The capability of binding methylated DNA can be tested by methods known in the art. The term “polypeptide” when used herein means a peptide, a protein, or a polypeptide which are used interchangeable and which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. As mentioned the terms polypeptide and protein are often used interchangeably herein. The term polypeptide also refers to, and does not exclude, modifications of the polypeptide. Modifications include glycosylation, acetylation, acylation, phosphorylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination; see, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York (1983), pgs. 1-12; Seifter, Meth. Enzymol. 182 (1990); 626-646, Rattan, Ann. NY Acad. Sci. 663 (1992); 48-62. Preferably, the term “polypeptide” encompasses a polypeptide capable of binding methylated DNA. Said term also encompasses a bifunctional polypeptide which is capable of binding methylated DNA and it encompasses an anti-methylated DNA antibody. Such polypeptides are described herein and are employed in the method of the present invention for detecting methylated DNA.

A “bifunctional polypeptide” means that a polypeptide has, in addition to binding to methylated DNA, preferably to CpG methylated DNA, due to an Fc portion of an antibody which is part of the said bifunctional polypeptide, further capabilities. For example, said Fc portion preferably offers the possibility to conjugate, link or covalently couple (a) compound(s) or moieties to said Fc portion. As used herein, the term “covalently coupled” means that the specified compounds or moieties are either directly covalently bonded to one another, or else are indirectly covalently joined to one another through an intervening moiety or moieties, such as a bridge, spacer, or linkage moiety or moieties. Furthermore, said Fc portion may be used to couple said bifunctional polypeptide to a container as is described herein. A preferred bifunctional polypeptide is characterized by the amino acid sequence shown in SEQ ID NO: 2 (FIG. 7). Further preferred bifunctional polypeptides are described herein below.

Without being bound by theory, it is believed that the nascent bifunctional polypeptide comprising an methyl-DNA-binding domain and an Fc portion of an antibody is folded within a host cell such that preferably two polypeptides are joined at their Fc portion in a manner similar or, preferably, identical to the constant region of an antibody, resulting in a bifunctional polypeptide as described herein.

It was surprisingly found that said bifunctional polypeptide, preferably behaving as an antibody-like protein can preferably bind CpG methylated DNA in an antibody-like manner. That means, the bifunctional polypeptide has a high affinity and high avidity to its “antigen” which is preferably methylated DNA that is preferably methylated at CpG dinucleotides. Again, without being bound by theory, the high affinity and avidity of the bifunctional polypeptide employed in the method of the present invention for detecting methylated DNA for its “antigen” is caused by the unique structure of said bifunctional polypeptide. This is because, it is assumed that the constant regions form disulfide-bonds between immunoglobulin heavy chains of the constant regions of each of two polypeptide molecules of said bifunctional polypeptide. Accordingly, preferably an antibody-like structure is formed which closely resembles the structure of an antibody.

Moreover, without being bound by theory it is assumed that this antibody-like structure lends, for example, stability on the bifunctional polypeptide employed in the method of the present invention for detecting methylated DNA. This is because, it is described in the art that proteins fused to a constant region of an antibody may confer a higher stability and half-life of the said protein. In addition, it is believed that the antibody-like structure caused by the intermolecular interaction of the constant regions brings the methyl-DNA-binding domain of one polypeptide of the bifunctional polypeptide used in accordance with the method of the present invention for detecting methylated DNA in close proximity to the methyl-DNA-binding domain of another polypeptide of the present invention employed in the method of the present invention. This allows bivalent interactions between the methyl-DNA-binding protein(s) and methylated DNA. Accordingly, the bifunctional polypeptide described herein is preferably capable of binding to its antigen via two methyl DNA-binding domains which are part of said bifunctional polypeptide. The high affinity binding of the bifunctional polypeptide is, inter alia, also achieved by using preferably methyl-DNA-binding domains of proteins instead of the full-length methyl-DNA-binding protein containing domains for the interaction with other proteins that may, however, disturb or interfere the unique applicability as described herein which are known to specifically bind to methylated DNA, preferably, CpG methylated DNA, rather than to unmethylated DNA. The preferred use of the methyl-DNA-binding domain, moreover, is believed to guarantee that indeed methylated DNA is bound since the detection is direct and not indirect. Most prior art methods can only indirectly detect methylated DNA by PCR.

These properties award the preferred bifunctional polypeptide to be a reliable and easy applicable diagnostic tool which can be employed in the method of the present invention for detecting methylated DNA. Yet, it can also be employed in methods for, inter alia, isolating, purifying enriching methylated DNA even if said DNA is only present in very small amounts, e.g., about more than 10 ng, less than 10 ng, less than 7.5 ng, less than 5 ng, less than 2.5 ng, less than 1000 pg, less than 500 pg, less than 250 pg or about 150 pg as described herein. Accordingly, due to its antibody-like structure the bifunctional polypeptide described herein is a robust molecule rendering it to be applicable, for instance, for various applications including multi-step procedures in a single tube assay as is described herein and in the appended Examples.

The term “contacting” includes every technique which causes that a polypeptide which is capable of binding methylated DNA as is described herein is brought into contact with a sample comprising methylated and/or unmethylated DNA. Preferably, said sample comprising methylated and/or unmethylated DNA is transferred preferably by a pipetting step into the container which is coated with a polypeptide described herein which is capable of binding methylated DNA.

A further advantage of the method of the present invention for detecting methylated DNA is that after the container, preferably a PCR tube has been coated, methylated DNA can be bound by a polypeptide which is capable of binding methylated DNA preferably within 40-50 minutes. Subsequent washing steps which are preferably applied only need preferably about 5 minutes which renders the herein described method for detecting methylated DNA a fast and robust method which can be run in a high-throughput format that can optionally be automated.

The term “detecting” encompasses any technique which is suitable for detecting methylated DNA.

In a preferred embodiment the methylated DNA bound by a polypeptide capable of binding methylated DNA is detected by restriction enzyme digestion, bisulfate sequencing, pyrosequencing or Southern Blot. However, the detection of methylated DNA is not limited to the aforementioned methods but includes all other suitable methods known in the art for detecting methylated DNA such as RDA, microarrays and the like. The term “methylated DNA” encompasses preferably methylated DNA, more preferably, CpG methylated DNA including hemi-methylated DNA or DNA methylated at both strands or single-stranded methylated DNA. The most important example is methylated cytosine that occurs mostly in the context of the dinucleotide CpG, but also in the context of CpNpG- and CpNpN-sequences. In principle, however, other naturally occurring nucleotides may also be methylated.

In an alternative, but also preferred embodiment the methylated DNA bound by a polypeptide capable of binding methylated DNA is detected by an amplification technique, preferably PCR, for example, conventional or real-time PCR including either single or multiplex conventional or real-time PCR using preferably gene-specific primers.

The term “amplification technique” refers to any method that allows the generation of a multitude of identical or essentially identical (i.e. at least 95% more preferred at least 98%, even more preferred at least 99% and most preferred at least 99.5% such as 99.9% identical) nucleic acid molecules or parts thereof. Such methods are well established in the art; see Sambrook et al. “Molecular Cloning, A Laboratory Manual”, 2^{nd} edition 1989, CSH Press, Cold Spring Harbor. Various PCR techniques, including real-time PCR are reviewed, for example, by Ding, J. Biochem. Mol. Biol. 37 (2004), 1-10.

As mentioned above, a variety of amplification methods are known in the art, all of which are expected to be useful for detecting methylated DNA bound by a polypeptide described herein which is capable of binding methylated DNA in the method of the invention. It is preferred that the detection in step (c) is effected by PCR. PCR is a powerful technique used to amplify DNA millions of fold by repeated replication of a template in a short period of time. The process utilizes sets of specific in vitro synthesized oligonucleotides to prime DNA synthesis. The design of the primers is dependent upon the sequences of the DNA that is desired to be analyzed. It is known that the length of a primer results from different parameters (Gillam (1979), Gene 8, 81-97; Innis (1990), PCR Protocols: A guide to methods and applications, Academic Press, San Diego, USA). Preferably, the primer should only hybridize or bind to a specific region of a target nucleotide sequence. The length of a primer that statistically hybridizes only to one region of a target nucleotide sequence can be calculated by the following formula: (¼)×(whereby x is the length of the primer). For example a hepta- or octanucleotide would be sufficient to bind statistically only once on a sequence of 37 kb. However, it is known that a primer exactly matching to a complementary template strand must be at least 9 base pairs in length, otherwise no stable-double strand can be generated (Goulian (1973), Biochemistry 12, 2893-2901). It is also envisaged that computer-based algorithms can be used to design primers capable of amplifying the nucleic acid molecules of the invention. Preferably, the primers of the invention are at least 10 nucleotides in length, more preferred at least 12 nucleotides in length, even more preferred at least 15 nucleotides in length, particularly preferred at least 18 nucleotides in length, even more particularly preferred at least 20 nucleotides in length, and most preferably at least 25 nucleotides in length. The invention, however, can also be carried out with primers which are shorter or longer.

The PCR technique is carried out through many cycles (usually 20-50) of melting the template at a high temperature, allowing the primers to anneal to complimentary sequences within the template and then replicating the template with DNA polymerase. The process has been automated with the use of thermostable DNA polymerases isolated from bacteria that grow in thermal vents in the ocean or hot springs. During the first round of replication a single copy of DNA is converted to two copies and so on resulting in an exponential increase in the number of copies of the sequences targeted by the primers. After just 20 cycles a single copy of DNA is amplified over 2,000,000 fold.

In a preferred embodiment, the aforementioned method further comprises step (d) analyzing the DNA bound by a polypeptide capable of binding to methylated DNA. The analysis is preferably done by sequencing. Said sequencing is preferably performed by methods known in the art, for example, automated didesoxy-sequencing using fluorescent didesoxy nucleotides according to the method of Sanger (Proc. Natl. Acad. Sci. 74 (1977), 5463-5467). For automated sequencing, the DNA to be sequenced is prepared according to methods known in the art and preferably according to the instructions of the kit used for preparing said DNA for sequencing.

Before the present invention is described in detail, it is to be understood that this invention is not limited to the particular methodology, protocols, bacteria, vectors, and reagents etc. described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland). Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integer or step.

It must be noted that as used herein and in the appended claims, the singular forms “a,” “an,” and “the,” include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

CpG islands frequently contain gene promoters and transcription start sites and are usually unmethylated in normal cells. Methylation of CpG-islands is associated with transcriptional repression. In cancer, the methylation of CpG-island promoters leads to the abnormal silencing of tumor-suppressor genes, thus contributing to the pathogenesis of the disease. As mentioned above, the prior art describes different approaches for the detection of methylated candidate genes which, however, suffer from certain shortcomings. For example, high throughput methods of the prior art may be limited to the detection of single/few CpG residues or may not reliable detect CpG methylated DNA, particularly if only low amounts of DNA can be made subject of an analysis. To allow a rapid and sensitive detection of the degree of CpG-methylation of candidate genes, the present invention provides means and methods that allow the detection of CpG-methylation, without applying, for example, methylation-sensitive restriction endonucleases or bisulfite-treatment.

In addition to the surprising finding mentioned herein above as regards the method of the present invention for detecting methylated DNA, it was further surprisingly found that binding of methylated DNA and/or fragments thereof to the relatively small surface of containers, preferably PCR-tubes is sufficient to detect preferably a single gene locus within a complex mixture of methylated and/or methylated DNA and/or fragments thereof. Accordingly, it was found that preferably a one-tube assay for detecting methylated DNA termed methyl-binding (MB)-PCR is a reliable and easy applicable diagnostic tool for, inter alia, isolating, purifying, enriching, and/or preferably detecting methylated DNA even if said DNA is only present in very small amounts, e.g., about more than 10 ng, less than 10 ng, less than 7.5 ng, less than 5 ng, less than 2.5 ng, less than 1000 pg, less than 500 pg, less than 250 pg or about 150 pg as described herein. Using the methods and kits described herein, it is possible to generate methylation profiles of single or multiple gene loci in, for example, human cancer in large numbers of samples.

Briefly, a preferred embodiment of the method of the present invention for detecting methylated DNA is MB-PCR which may work as follows:

A protein with preferably high affinity for methylated DNA, in particular for CpG-methylated DNA, is coated onto the walls of preferably a PCR-cycler compatible reaction container, preferably a tube and used to selectively capture methylated DNA and/or DNA-fragments from preferably a genomic DNA mixture. The retention of a specific DNA and/or DNA-fragment (e.g. a CpG island promoter of a specific gene) can be detected in the same container using PCR (either standard PCR or real-time PCR, single or multiplex). The degree of methylation may be estimated relative to a PCR reaction of the genomic input DNA. Thus, the present invention provides a quick, simple, reliable, robust, and extremely sensitive technique allowing the detection of methylated DNA, in particular, in tumorous tissue or tumor cells from limited samples.

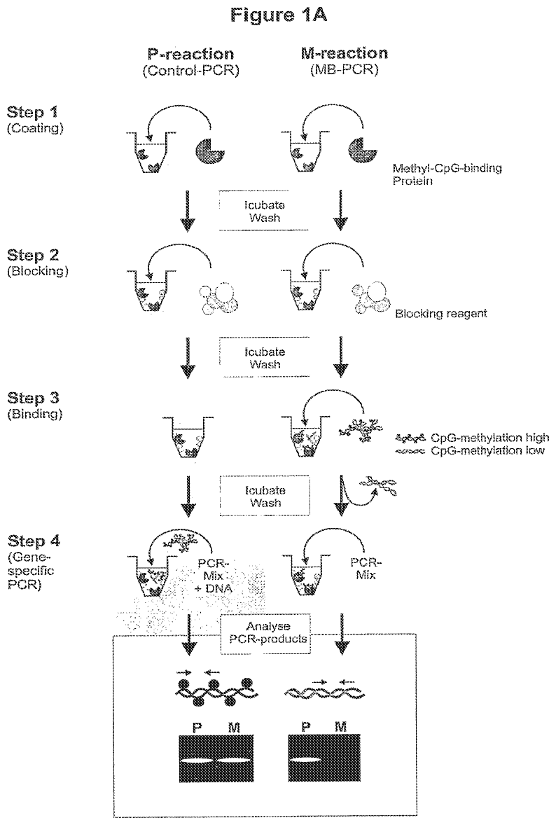

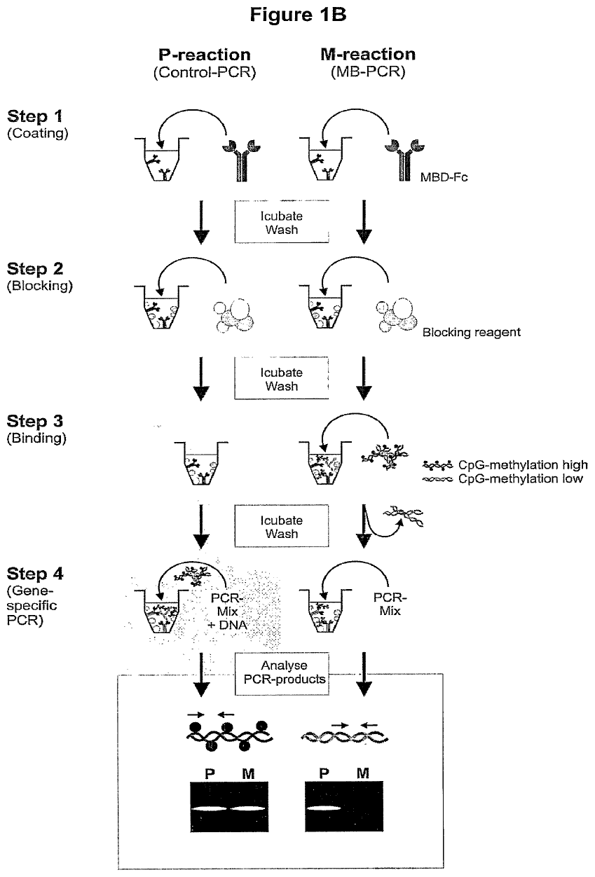

The preferred diagnostic application employing a polypeptide capable of binding methylated DNA is shown in FIG. 1A. FIG. 1B shows the preferred diagnostic application by employing a bifunctional polypeptide which is capable of binding CpG-methylated DNA as described herein. Briefly, in a first step preferably a methyl-CpG-binding polypeptide is preferably added into a coatable PCR-vessel, for example, TopYield™ Strips from Nunc. In doing so, the polypeptide is preferably coated onto the inner surface of said vessel by techniques known in the art and described herein. In a next step, blocking reagents, e.g., about 5% milk powder are added into the coated PCR vessel. In a further step, preferably DNA-fragments of interest (for example, methylated and/or unmethylated DNA-fragments (the term “CpG-methylation low” used in FIGS. 1A and 1B comprises and particularly refers to unmethylated DNA)) are added into the coated and blocked PCR vessel. It is believed that the methyl-CpG-binding polypeptide binds specifically to methylated DNA, if present. In a following step, the coated and blocked PCR vessel containing preferably DNA-fragments is incubated and then washed to remove unbound DNA-fragments. Afterwards, a PCR mix including preferably gene-specific primers or, but also preferred, at least two, three, four, five, six, seven etc. pairs of primers for, e.g., multiplex PCR for the gene or genlocus or genloci of interest which is/are suspected to be methylated or unmethylated is added to run preferably, a real time PCR or conventional PCR followed by gel electrophoresis to separate amplification products. Optionally, a control reaction can be performed as is shown in FIG. 1A or 1B as “P-reaction” which is described herein below.

A preferred detailed protocol for MB-PCR is as follows:

Preferably, the PCR tubes are prepared using heat stable TopYield™ Strips (Nunc Cat. No. 248909). Preferably, 50 μl of the a polypeptide described herein, preferably a methyl-CpG-binding polypeptide (diluted at 15 μg/ml in 10 mM Tris/HCl pH 7.5) are added to each well and incubated overnight at 4° C. Preferably, wells are washed three times with 200 μl TBS (20 mM Tris, pH 7.4 containing 170 mM NaCl) and blocked preferably for 3-4 hr at RT with 100 μl Blocking Solution (10 mM Tris, pH 7.5 containing 170 mM NaCl, 5% skim milk powder, 5 mM EDTA and 1 μg/ml of each poly d(I/C), poly d(A/T) and poly d(CG)). Preferably, tubes are then washed three times with 200 μl TBST (TBS containing 0.05% Tween-20).

Preferably, 50 μl Binding Buffer (20 mM Tris, pH 7.5 containing 400 mM NaCl, 2 mM MgCl₂, 0.5 mM EDTA, and 0.05% Tween-20) are added to each well and preferably 2 μl of digested DNA, preferably genomic DNA digested with MseI in an amount of preferably 5 ng/μl is added to every second well (M-reaction).

Genomic DNA is preferably prepared by using a kit known in the art, for example, using Blood and Cell Culture Midi Kit (Qiagen). The quality of the genomic DNA-preparation is preferably controlled by agarose gel electrophoresis and DNA concentration was preferably determined by UV spectrophotometry. Quantitation of DNA is preferably done by using PicoGreen dsDNA Quantitation Reagent (Molecular Probes).

The wells containing a polypeptide described herein and DNA, preferably DNA-fragments (generated by enzymatic digestion or mechanically fragmented), are incubated on a shaker at preferably RT for preferably 40-50 min. Preferably, tubes were washed two times with 200 μl Binding Buffer and once with 10 mM Tris/HCl pH 8.0.

Next, PCR is preferably carried out directly in the TopYield™ Strips. Preferably, the PCR-Mix (50 μl/well), preferably PCR Master Mix (Promega), contains preferably 10 pmol of each gene-specific primer (synthesized by Metabion). Primer sequences and cycling parameters for specific genes of interest are given in the Example. Of course, any other suitable gene specific or genlocus specific or genloci specific primers can be designed by the person skilled in the art. Moreover, the skilled artisan can readily determine and/or test the PCR parameters most suitable for the primer(s) and gene(s), genlocus/genloci of interest. After adding the PCR-mix, preferably 1 μl Mse I-digested DNA (preferably in an amount of 5 ng/μl) is added to every second other well that was not previously incubated with DNA-fragments (P-reaction). Preferably, PCR-products are analyzed using agarose gel electrophoresis and the ethidium bromide stained gel was scanned using, for example, a Typhoon 9200 Imager (Amersham/Pharmacia).

Optionally, a control reaction can be performed as is shown in FIG. 1A or 1B as “P-reaction” which is described herein below.

Accordingly, it is envisaged that the method of the present invention is useful for the detection of methylated DNA, preferably CpG-methylated DNA, in a sample as described herein below which may include (a) single cell(s). It is also envisaged to be useful for whole cells. “Whole cell” means the genomic context of a whole single cell.

In the following, preferred polypeptides capable of binding methylated DNA are described. Accordingly, a polypeptide used in the methods of the present invention for detecting methylated DNA is preferably selected from the group consisting of
(a) a polypeptide belonging to the Methyl-DNA binding protein (MBD) family;
(b) a fragment of the polypeptide of (a), wherein said fragment is capable of binding methylated DNA;
(c) a variant of the polypeptide of (a) or the fragment of (b), wherein in said variant one or more amino acid residues are substituted compared to the polypeptide of (a) or the fragment of (b), and wherein said variant is capable of binding methylated DNA;
(d) a polypeptide which is an anti-methylated DNA antibody or fragment thereof; and
(e) a polypeptide which is at least 70% identical to a polypeptide of any one of (a) to (c) and which is capable of binding methylated DNA.

Of course, it is envisaged that the herein described polypeptides which are capable of detecting methylated DNA are encoded by a nucleic acid molecule. The term “nucleic acid molecule” when used herein encompasses any nucleic acid molecule having a nucleotide sequence of bases comprising purine and pyrimidine bases which are comprised by said nucleic acid molecule, whereby said bases represent the primary structure of a nucleic acid molecule. Nucleic acid sequences include DNA, cDNA, genomic DNA, RNA, synthetic forms, for example, PNA, and mixed polymers, both sense and antisense strands, or may contain non-natural or derivatized nucleotide bases, as will be readily appreciated by those skilled in the art. The polynucleotide of the present invention encoding a polypeptide which is capable of binding methylated DNA and which is employed in the method of the present invention is preferably composed of any polyribonucleotide or polydeoxyribonucleotide, which may be unmodified RNA or DNA or modified RNA or DNA. For example, the polynucleotide can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. The polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, the term “nucleic acid molecules” embraces chemically, enzymatically, or metabolically modified forms.

In an alternative, but also preferred embodiment, a bifunctional polypeptide (i.e. the MBD protein to be employed in the methods and kits provided herein) capable of binding methylated DNA which is employed in the method of the present invention is encoded by a nucleic acid molecule comprising a nucleotide sequence of the present invention described hereinabove is selected from the group consisting of:
(a) a nucleic acid sequence having the nucleotide sequence shown in SEQ ID NO: 1 (FIG. 7);
(b) a nucleic acid sequence having a nucleotide sequence encoding a polypeptide having the amino acid sequence shown in SEQ ID: NO 2 (FIG. 7);
(c) a nucleic acid sequence having a nucleotide sequence encoding a fragment of a polypeptide having the amino acid sequence shown in SEQ ID: NO 2 (FIG. 7), wherein said fragment comprises at least amino acids 130 to 361 of said polypeptide and which is capable of binding methylated DNA;
(d) a nucleic acid sequence having a nucleotide sequence encoding a variant of a polypeptide encoded by a polynucleotide of any one of (a) to (c), wherein in said variant one or more amino acid residues are substituted compared to said polypeptide, and said variant is capable of binding methylated DNA;
(e) a nucleic acid sequence having a nucleotide sequence which hybridizes with a nucleic acid sequence of any one of (a) to (d) and which is at least 65% identical to the nucleotide sequence of the nucleic acid molecule of (a) and which encodes a polypeptide being capable of binding methylated DNA;
(f) a nucleic acid molecule encoding a polypeptide which is at least 65% identified to a polypeptide encoded by a nucleic acid molecule of (b) and which is capable of binding methylated DNA; and
(g) a nucleic acid sequence having a nucleotide sequence being degenerate to the nucleotide sequence of the polynucleotide of any one of (a) to (f);
   or the complementary strand of such a polynucleotide.

The above embodiment relates, accordingly, e.g. to the use of a “MBD-Fc” molecule in the kits and methods provided herein.

As described above, a fragment of a bifunctional polypeptide employed in the method of the present invention for detecting methylated DNA which has the amino acid sequence shown in SEQ ID: NO 2 (FIG. 7) comprises at least amino acids 130 to 361 of the amino acid sequence shown in SEQ ID: NO 2 (FIG. 7). That means that said fragment may comprise in addition to amino acids 130 to 361 which represent the Fc portion, one or more amino acids such that said fragment is capable of binding methylated DNA, preferably, CpG methylated DNA, rather than unmethylated DNA. Accordingly, it is envisaged that said fragment comprises more preferably, at least amino acids 116 to 361 of the amino acid sequence shown in SEQ ID: NO 2 (FIG. 7). Even more preferably, said fragment may comprise at least amino acids 29 to 115 and 130 to 361 of the amino acid sequence shown in SEQ ID: NO 2 (FIG. 7). In a most preferred embodiment, said fragment may comprise at least amino acids 29 to 361. It is generally preferred that the fragments of the a polypeptide described herein are able to bind to methylated DNA, preferably to CpG methylated DNA, rather than unmethylated DNA. This ability can be tested by methods known in the art or preferably by those methods described in the appended Examples.

The present invention preferably also relates to methods, wherein nucleic acid sequences which hybridize to the nucleic acid sequence encoding a polypeptide which is capable of binding methylated DNA are employed. Said hybridizing nucleic acids encode a polypeptide which is capable of binding methylated DNA: Moreover, in the methods of the present invention, nucleic acids are employed which hybridize to the sequences shown in SEQ ID NO: 1 or fragments or variants thereof as described herein (FIG. 7) and which are at least 65% identical to the nucleic acid sequence shown in SEQ ID NO: 1 (FIG. 7) and which preferably encode a bifunctional polypeptide being capable of binding methylated DNA, preferably CpG methylated DNA, rather than unmethylated DNA, wherein said polypeptide is employed in the method of the present invention for detecting methylated DNA. Furthermore, the present invention preferably relates to methods in which nucleic acid sequences encoding a polypeptide are employed which are at least 65%, more preferably 70%, 75%, 80%, 85%, 90%, more preferably 99% identical to a polypeptide as described herein which is capable of binding methylated DNA. It is also preferably envisaged that in the methods of the present invention polypeptides are employed which are at least 65%, more preferably 70%, 75%, 80%, 85%, 90%, more preferably 99% identical to the polypeptide shown in SEQ ID NO:2. The term “hybridizes” as used in accordance with the present invention preferably relates to hybridizations under stringent conditions. The term “hybridizing sequences” preferably refers to sequences which display a sequence identity of at least 65%, even more preferably at least 70%, particularly preferred at least 80%, more particularly preferred at least 90%, even more particularly preferred at least 95% and most preferably at least 97, 98% or 99% identity with a nucleic acid sequence as described above encoding a polypeptide which is capable of binding methylated DNA or a bifunctional polypeptide which is able to bind to methylated DNA, preferably CpG methylated DNA, rather than unmethylated DNA, wherein said polypeptide capable of binding methylated DNA or said bifunctional polypeptide is employed in the method of the present invention for detecting methylated DNA.

Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above due to problems with compatibility. Hybridizing nucleic acid molecules also comprise fragments of the above described molecules. Such fragments may represent nucleic acid sequences as described herein. Furthermore, nucleic acid molecules which hybridize with any of the aforementioned nucleic acid molecules also include complementary fragments, derivatives, and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial,” in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

Moreover, the present invention also relates to methods employed nucleic acid molecules the sequence of which is degenerate in comparison with the sequence of an above-described nucleic acid molecules, wherein such degenerate nucleic acid molecules encode a polypeptide which is capable of binding methylated DNA or which encode a bifunctional polypeptide as described herein and which is employed in the method of the present invention for detecting methylated DNA. When used in accordance with the present invention the term “being degenerate as a result of the genetic code” means that due to the redundancy of the genetic code different nucleotide sequences code for the same amino acid.

Of course, the present invention also envisages the complementary strand to the aforementioned and below mentioned nucleic acid molecules if they may be in a single-stranded form.

Preferably, the nucleic acid molecule encoding a polypeptide which is capable of binding methylated DNA or a bifunctional polypeptide capable of binding methylated DNA and which is/are employed in the method of the present invention may be any type of nucleic acid, e.g. DNA, genomic DNA, cDNA, RNA or PNA (peptide nucleic acid).

For the purposes of the present invention, a peptide nucleic acid (PNA) is a polyamide type of DNA analog and the monomeric units for adenine, guanine, thymine and cytosine are available commercially (Perceptive Biosystems). Certain components of DNA, such as phosphorus, phosphorus oxides, or deoxyribose derivatives, are not present in PNAs. As disclosed by Nielsen et al., Science 254:1497 (1991); and Egholm et al., Nature 365:666 (1993), PNAs bind specifically and tightly to complementary DNA strands and are not degraded by nucleases. In fact, PNA binds more strongly to DNA than DNA itself does. This is probably because there is no electrostatic repulsion between the two strands, and also the polyamide backbone is more flexible. Because of this, PNA/DNA duplexes bind under a wider range of stringency conditions than DNA/DNA duplexes making it easier to perform multiplex hybridization. Smaller probes can be used than with DNA due to the strong binding. In addition, it is more likely that single base mismatches can be determined with PNA/DNA hybridization because a single mismatch in a PNA/DNA 15-mer lowers the melting point (T.sub.m) by 8°−20° C., vs. 4°−16° C. for the DNA/DNA 15-mer duplex. Also, the absence of charge groups in PNA means that hybridization can be done at low ionic strengths and reduce possible interference by salt during the analysis.

The DNA may, for example, be genomic DNA or cDNA. The RNA may be, e.g., mRNA. The nucleic acid molecule may be natural, synthetic, or semisynthetic, or it may be a derivative, such as peptide nucleic acid (Nielsen, Science 254 (1991), 1497-1500) or phosphorothioates. Furthermore, the nucleic acid molecule may be a recombinantly produced chimeric nucleic acid molecule comprising any of the aforementioned nucleic acid molecules either alone or in combination.

The nucleic acid molecule encoding a polypeptide described herein which is employed in the method of the present invention for detecting methylated DNA is envisaged to be contained in a vector (e.g. a plasmid, cosmid, virus, or bacteriophage) which may be transformed into a host cell (a prokaryotic or eukaryotic cell) so as to, inter alia, produce a polypeptide of the present invention which is employed in the method of the present invention. A polypeptide of the invention which is employed in the method of the present invention may be produced by microbiological methods or by transgenic mammals. It is also envisaged that a polypeptide of the invention is recovered from transgenic plants. Alternatively, a polypeptide of the invention may be produced synthetically or semi-synthetically.

Preferably, the nucleic acid molecule of the present invention is part of a vector. Therefore, the present invention relates in another embodiment to a vector comprising the nucleic acid molecule of this invention. Such a vector may be, e.g., a plasmid, cosmid, virus, bacteriophage or another vector used e.g. conventionally in genetic engineering, and may comprise further genes such as marker genes which allow for the selection and/or replication of said vector in a suitable host cell and under suitable conditions. In a preferred embodiment, said vector is an expression vector, in which the nucleic acid molecule of the present invention is operatively linked and to expression control sequence(s) allowing expression in prokaryotic or eukaryotic host cells as described herein. The term “operatively linked,” as used in this context, refers to a linkage between one or more expression control sequences and the coding region in the polynucleotide to be expressed in such a way that expression is achieved under conditions compatible with the expression control sequence.

The nucleic acid molecules of the present invention may thus be inserted into several commercially available vectors. Non-limiting examples include plasmid vectors compatible with mammalian cells, such as pUC, pBluescript (Stratagene), pET (Novagen), pREP (Invitrogen), pCRTopo (Invitrogen), pcDNA3 (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, pUCTag, pIZD35, pLXIN and pSIR (Clontech), and plRES-EGFP (Clontech). Preferably, the nucleic acid molecules of the present invention are inserted into the vector Signal pIG plus (Ingenius, R&D Systems). Baculovirus vectors such as pBlueBac, BacPacz Baculovirus Expression System (CLONTECH) and MaxBac™ Baculovirus Expression System insect cells and protocols (Invitrogen) are available commercially and may also be used to produce high yields of biologically active protein. (see also, Miller (1993), Curr. Op. Genet. Dev., 3, 9; O'Reilly, Baculovirus Expression Vectors: A Laboratory Manual, p. 127). In addition, prokaryotic vectors such as pcDNA2; and yeast vectors such as pYes2 are non-limiting examples of other vectors suitable for use with the present invention.

Other preferred expression vectors of the present application are those for expressing proteins in Drosophila cells which are well known in the art, such as the DES®-series of Invitrogen. Preferably, said Drosophila cell expression vector is pMTBiP/V5-His B (Invitrogen). The pMT/BiP/V5-His vector offers the following additional features. It has a small size (3.6 kb) to improve DNA yields and increase subcloning efficiency, it has a C-terminal V5 epitope tag for rapid detection with Anti-V5 Antibody, and it has a C-terminal 6×His tag for simple purification of recombinant fusion proteins using nickel-chelating resin.

For vector modification techniques, see Sambrook and Russel (2001), loc. cit. Vectors can contain one or more replication and inheritance systems for cloning or expression, one or more markers for selection in the host, e. g., antibiotic resistance, and one or more expression cassettes.

The coding sequences inserted in the vector can be synthesized by standard methods, isolated from natural sources, or prepared as hybrids. Ligation of the coding sequences to transcriptional regulatory elements (e.g., promoters, enhancers, and/or insulators) and/or to other amino acid encoding sequences can be carried out using established methods.

Furthermore, the vectors may, in addition to the nucleic acid sequences of the invention, comprise expression control elements, allowing proper expression of the coding regions in suitable hosts. Such control elements are known to the artisan and may include a promoter, translation initiation codon, translation, and insertion site or internal ribosomal entry sites (IRES) (Owens, Proc. Natl. Acad. Sci. USA 98 (2001), 1471-1476) for introducing an insert into the vector. Preferably, the nucleic acid molecule of the invention is operatively linked to said expression control sequences allowing expression in eukaryotic or prokaryotic cells.

Control elements ensuring expression in eukaryotic and prokaryotic cells are well known to those skilled in the art. As mentioned above, they usually comprise regulatory sequences ensuring initiation of transcription and optionally poly-A signals ensuring termination of transcription and stabilization of the transcript. Additional regulatory elements may include transcriptional, as well as translational enhancers, and/or naturally-associated or heterologous promoter regions. Possible regulatory elements permitting expression in for example mammalian host cells comprise the CMV-HSV thymidine kinase promoter, SV40, RSV-promoter (Rous sarcome virus), human elongation factor 1α-promoter, CMV enhancer, CaM-kinase promoter or SV40-enhancer.

For the expression in prokaryotic cells, a multitude of promoters including, for example, the tac-lac-promoter, the lacUV5 or the trp promoter, has been described. Beside elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-Vitrogene, as used, inter alia in the appended examples), pSPORT1 (GIBCO BRL) or pGEMHE (Promega), or prokaryotic expression vectors, such as lambda gt11.

An expression vector according to this invention is at least capable of directing the replication, and preferably the expression, of the nucleic acids and protein of this invention. Suitable origins of replication include, for example, the Col E1, the SV40 viral and the M 13 origins of replication. Suitable promoters include, for example, the cytomegalovirus (CMV) promoter, the lacZ promoter, the gal10 promoter, and the Autographa californica multiple nuclear polyhedrosis virus (AcMNPV) polyhedral promoter. Suitable termination sequences include, for example, the bovine growth hormone, SV40, lacZ and AcMNPV polyhedral polyadenylation signals. Examples of selectable markers include neomycin, ampicillin, and hygromycin resistance and the like. Specifically-designed vectors allow the shuttling of DNA between different host cells, such as bacteria-yeast, bacteria-animal cells, bacteria-fungal cells, or bacteria invertebrate cells.

Beside the nucleic acid molecules of the present invention, the vector may further comprise nucleic acid sequences encoding for secretion signals. The secretion signal of the present invention that is preferably used in accordance with the present invention when the polypeptide of the present invention is expressed in Drosophila cells, preferably Drosophila S2 cells is the Drosophila BiP secretion signal well known in the art. The preferred BiP secretion signal that is used in the context of the present invention is shown in the amino acid sequence of SEQ ID NO: 2 at positions 1 to 28. Other secretion signal sequences are well known to the person skilled in the art. Furthermore, depending on the expression system used leader sequences capable of directing the expressed polypeptide to a cellular compartment may be added to the coding sequence of the nucleic acid molecules of the invention and are well known in the art. The leader sequence(s) is (are) assembled in appropriate phase with translation, initiation and termination sequences, and preferably, a leader sequence capable of directing secretion of translated protein or a part thereof, into, inter alia, the extracellular membrane. Optionally, the heterologous sequence can encode a fusion protein including an C- or N-terminal identification peptide imparting desired characteristics, e.g., stabilization or simplified purification of expressed recombinant product. Once the vector has been incorporated into the appropriate host, the host is maintained under conditions suitable for high level expression of the nucleotide sequences, and, as desired, the collection and purification of the proteins, antigenic fragments, or fusion proteins of the invention may follow. Of course, the vector can also comprise regulatory regions from pathogenic organisms.

Furthermore, said vector may also be, besides an expression vector, a gene transfer and/or gene targeting vector. Gene therapy, which is based on introducing therapeutic genes (for example for vaccination) into cells by ex-vivo or in-vivo techniques, is one of the most important applications of gene transfer. Suitable vectors, vector systems and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhlhauser, Circ. Res. 77 (1995), 1077-1086; Wang, Nature Medicine 2 (1996), 714-716; WO 94/29469; WO 97/00957; Schaper, Current Opinion in Biotechnology 7 (1996), 635-640 or Verma, Nature 389 (1997), 239-242 and references cited therein.

The nucleic acid molecules of the invention and vectors as described herein above may be designed for direct introduction or for introduction via liposomes or viral vectors (e.g. adenoviral, retroviral) into the cell. Additionally, baculoviral systems or systems based on vaccinia virus or Semliki Forest Virus can be used as eukaryotic expression system for the nucleic acid molecules of the invention. In addition to recombinant production, fragments of the protein, the fusion protein or antigenic fragments of the invention may be produced by direct peptide synthesis using solid-phase techniques (cf Stewart et al. (1969) Solid Phase Peptide Synthesis; Freeman Co, San Francisco; Merrifield, J. Am. Chem. Soc. 85 (1963), 2149-2154). In vitro protein synthesis may be performed using manual techniques or by automation. Automated synthesis may be achieved, for example, using Applied Biosystems 431A Peptide Synthesizer (Perkin Elmer, Foster City Calif.) in accordance with the instructions provided by the manufacturer. Various fragments may be chemically synthesized separately and combined using chemical methods to produce the full length molecule.

The present invention, in addition, relates to a host cell genetically engineered with the nucleic acid molecule of the invention or a vector of the present invention. Said host may be produced by introducing said vector or nucleotide sequence into a host cell which upon its presence in the cell mediates the expression of a protein encoded by the nucleotide sequence of the invention or comprising a nucleotide sequence or a vector according to the invention wherein the nucleotide sequence and/or the encoded polypeptide is foreign to the host cell.

By “foreign,” it is meant that the nucleotide sequence and/or the encoded polypeptide is either heterologous with respect to the host, this means derived from a cell or organism with a different genomic background or is homologous with respect to the host but located in a different genomic environment than the naturally occurring counterpart of said nucleotide sequence. This means that if the nucleotide sequence is homologous with respect to the host it is not located in its natural location in the genome of said host, in particular, it is surrounded by different genes. In this case the nucleotide sequence may be either under the control of its own promoter or under the control of a heterologous promoter. The location of the introduced nucleic acid molecule or the vector can be determined by the skilled person by using methods well-known to the person skilled in the art, e.g., Southern Blotting. The vector or nucleotide sequence according to the invention which is present in the host may either be integrated into the genome of the host or it may be maintained in some form extrachromosomally. In this respect, it is also to be understood that the nucleotide sequence of the invention can be used to restore or create a mutant gene via homologous recombination.

Said host may be any prokaryotic or eukaryotic cell. Suitable prokaryotic/bacterial cells are those generally used for cloning like E. coli, Salmonella typhimurium, Serratia marcescens, or Bacillus subtilis. Said eukaryotic host may be a mammalian cell, an amphibian cell, a fish cell, an insect cell, a fungal cell, a plant cell, or a bacterial cell (e.g., E. coli strains HB101, DH5a, XL1 Blue, Y1090, and JM101). Eukaryotic recombinant host cells are preferred. Examples of eukaryotic host cells include, but are not limited to, yeast, e.g., Saccharomyces cerevisiae, Schizosaccharomyces pombe, Kluyveromyces lactis, or Pichia pastoris cells, cell lines of human, bovine, porcine, monkey, and rodent origin, as well as insect cells, including but not limited to, Spodoptera frugiperda insect cells and zebra fish cells.

Drosophila cells, however, are preferred. More preferably, said Drosophila cells are Drosophila S2 (ATCC CRL-1963) which are, preferably used for heterologous protein expression in Drosophila expression systems, for example, the Drosophila Expression System (DES®). The S2 cell line was derived from a primary culture of late stage (20-24 hours old) Drosophila melanogaster embryos. This versatile cell line grows rapidly at room temperature without CO₂ and is easily adapted to suspension culture. Generally, when expressing the polypeptide of the present invention insect cells are preferred since they have the advantage that they contain less or, preferably, no methylated DNA. Accordingly, when expressing and isolating and preferably purifying the polypeptide of the present invention, said polypeptide is preferably not contaminated with methylated DNA to which it can preferably bind. Another advantage of using insect cells is that they grow preferably in a protein-free medium which thus minimizes a further contamination of the polypeptide of the present invention when isolating, recovering and/or purifying the polypeptide of the present invention from preferably culture medium if said polypeptide is preferably secreted into said culture medium. Mammalian species-derived cell lines suitable for use and commercially available include, but are not limited to, L cells, CV-1 cells, COS-1 cells (ATCC CRL 1650), COS-7 cells (ATCC CRL 1651), HeLa cells (ATCC CCL 2), C1271 (ATCC CRL 1616), BS-C-1 (ATCC CCL 26), and MRC-5 (ATCC CCL 171).

In another embodiment, the present invention relates to a method for producing a polypeptide which is capable of binding methylated DNA, preferably CpG methylated DNA comprising culturing the host cell of the invention and recovering the produced polypeptide. Said polypeptide is preferably encoded by a nucleic acid molecule of the invention.

The present invention also provides a process for producing cells capable of expressing a polypeptide of the present invention which is capable of binding methylated DNA, preferably CpG methylated DNA, comprising genetically engineering cells in vitro by methods known in the art or by those described herein. Said polypeptide is preferably encoded by a nucleic acid molecule of the present invention.

A large number of suitable methods exist in the art to produce polypeptides in appropriate hosts. If the host is a unicellular organism or a mammalian or insect cell, the person skilled in the art can revert to a variety of culture conditions that can be further optimized without an undue burden of work. Conveniently, the produced protein is harvested from the culture medium or from isolated (biological) membranes by established techniques. Furthermore, the produced polypeptide may be directly isolated from the host cell.

The polypeptide of the invention may be produced by microbiological methods or by transgenic mammals. It is also envisaged that the polypeptide of the invention is recovered from transgenic plants. Alternatively, the polypeptide of the invention may be produced synthetically or semi-synthetically.

For example, chemical synthesis, such as the solid phase procedure described by Houghton Proc. Natl. Acad. Sci. USA (82) (1985), 5131-5135, can be used. Another method is in vitro translation of mRNA. A preferred method involves the recombinant production of protein in host cells as described above. For example, nucleotide acid sequences comprising all or a portion of any one of the nucleotide sequences according to the invention can be synthesized by PCR, inserted into an expression vector, and a host cell transformed with the expression vector. Thereafter, the host cell is cultured to produce the desired polypeptide, which is isolated and purified. Protein isolation and purification can be achieved by any one of several known techniques; for example and without limitation, ion exchange chromatography, gel filtration chromatography and affinity chromatography, high pressure liquid chromatography (HPLC), and reversed phase HPLC, preparative disc gel electrophoresis. In addition, cell-free translation systems can be used to produce a polypeptides of the present invention. Suitable cell-free expression systems for use in accordance with the present invention include rabbit reticulocyte lysate, wheat germ extract, canine pancreatic microsomal membranes, E. coli S30 extract, and coupled transcription/translation systems such as the TNT-system (Promega). These systems allow the expression of recombinant polypeptides or peptides upon the addition of cloning vectors, DNA fragments, or RNA sequences containing coding regions and appropriate promoter elements. As mentioned supra, protein isolation/purification techniques may require modification of the proteins of the present invention using conventional methods. For example, a histidine tag can be added to the protein to allow purification on a nickel column. Other modifications may cause higher or lower activity, permit higher levels of protein production, or simplify purification of the protein. After production of a polypeptide, which is employed in the method of the present invention, it may be modified by pegylation, derivatization, and the like.

The term “polypeptide belonging to the Methyl-DNA binding protein (MBD)” encompasses a polypeptide which has preferably the structural and/or functional characteristics of the methyl-DNA-binding domain (MBD) of a protein of the MBD family which comprises the proteins MeCP2, MBD1, MBD2, MBD3, and MBD4. Said term also encompasses polypeptides with the capability of binding methylated DNA, including, inter alia, antibodies raised against methylated DNA. Preferably, said antibody is an anti-5-methylcysteine antibody or fragment thereof. Preferably, said fragment is a Fab, F(ab′)₂, Fv or scFv fragment. The methyl-DNA-binding activity can be tested by methods known in the art. It is preferred that a polypeptide described herein binds methylated DNA either as a monomer or dimer or multivalent molecule as described elsewhere herein. It is preferably capable of binding to highly methylated DNA or low methylated DNA. Preferably, it can bind single methylated CpG pairs. MeCP2, MBD1, MBD2, MBD3, and MBD4 constitute a family of vertebrate proteins that share the methyl-CpG-binding domain. The MBD protein family comprises two subgroups based upon sequences of the known MBDs. The methyl-DNA-binding domain of MBD4 is most similar to that of MeCP2 in primary sequence, while the methyl-DNA-binding domain of MBD1, MBD2, and MBD3 are more similar to each other than to those of either MBD4 or MeCP2. However, the methyl-DNA-binding domains within each protein appear to be related evolutionarily based on the presence of an intron located at a conserved position within all five genes of MeCP2, MBD1, MBD2, MBD3, and MBD4. Yet, the sequence similarity between the members of the MBD family is largely limited to their methyl-DNA-binding domain, although MBD2 and MBD3 are similar and share about 70% of overall identity over most of their length. The greatest divergence occurs at the C-terminus, where MBD3 has 12 consecutive glutamic acid residues.

A protein belonging to the MBD family or fragment thereof, preferably a methyl-DNA-binding domain, useful in accordance with the methods of the present invention can, for example, be identified by using sequence comparisons and/or alignments by employing means and methods known in the art, preferably those described herein and comparing and/or aligning (a) known MBD(s) to/with a sequence suspected to be an MBD.

For example, when a position in both of the two compared sequences is occupied by the same base or amino acid monomer subunit (for instance, if a position in each of the two DNA molecules is occupied by adenine, or a position in each of two polypeptides is occupied by a lysine), then the respective molecules are identical at that position. The percentage identity between two sequences is a function of the number of matching or identical positions shared by the two sequences divided by the number of positions compared×100. For instance, if 6 of 10 of the positions in two sequences are matched or are identical, then the two sequences are 60% identical. By way of example, the DNA sequences CTGACT and CAGGTT share 50% homology (3 of the 6 total positions are matched). Generally, a comparison is made when two sequences are aligned to give maximum homology and/or identity. Such alignment can be provided using, for instance, the method of Needleman, J. Mol Biol. 48 (1970): 443-453, implemented conveniently by computer programs such as the Align program (DNAstar, Inc.). Homologous sequences share identical or similar amino acid residues, where similar residues are conservative substitutions for or “allowed point mutations” of, corresponding amino acid residues in an aligned reference sequence. In this regard, a “conservative substitution” of a residue in a reference sequence are those substitutions that are physically or functionally similar to the corresponding reference residues, e. g., that have a similar size, shape, electric charge, or chemical properties, including the ability to form covalent or hydrogen bonds, or the like. Particularly preferred conservative substitutions are those fulfilling the criteria defined for an “accepted point mutation” in Dayhoff et al., 5: Atlas of Protein Sequence and Structure, 5: Suppl. 3, chapter 22: 354-352, Nat. Biomed. Res. Foundation, Washington, D.C. (1978).

Preferably, a fragment of a polypeptide described herein and employed in the method of the present invention which is capable of binding methylated DNA, preferably, a methyl-DNA-binding domain or fragment thereof of a polypeptide employed in the method of the present invention, has preferably the structural and/or functional characteristics of a protein belonging to the MBD-family as described herein. Preferably, a fragment of a methyl-DNA-binding protein described herein is able to bind methylated DNA, preferably CpG methylated DNA.

The methyl-DNA-binding domain or fragment thereof of a polypeptide of the present invention which is employed in the method of the present invention is preferably of insect origin, nematode origin, fish origin, amphibian origin, more preferably of vertebrate origin, even more preferably of mammal origin, most preferably of mouse, and particularly preferred of human origin.

Preferably, the methyl-DNA-binding domain or fragment thereof of a polypeptide of the present invention which is employed in the method of the present invention possesses a unique alpha-helix/beta-strand sandwich structure with characteristic loops as is shown in FIG. 1 of Ballester and Wolffe, Eur. J. Biochem. 268 (2001), 1-6 and is able to bind methylated DNA.

More preferably, the protein belonging to the MBD family or fragment thereof of a polypeptide of the present invention which is employed in the method of the present invention comprises at least 50, more preferably at least 60, even more preferably at least 70 or at least 80 amino acid residues of the MBDs shown in FIG. 1 of Ballester and Wolffe (2001), loc. cit. and is able to bind methylated DNA.

Even more preferably, the methyl-DNA-binding domain or fragment or variant thereof of a polypeptide of the present invention employed in the method of the present invention shares preferably 50%, 60%, 70%, 80% or 90%, more preferably 95% or 97%, even more preferably 98%, and most preferably 99% identity on amino acid level to the MBDs shown in FIG. 1 of Ballester and Wolffe (2001), loc. cit. and is able to bind methylated DNA. Means and methods for determining the identity of sequences, for example, amino acid sequences is described elsewhere herein.

In accordance with the present invention, the term “identical” or “percent identity” in the context of two or more nucleic acid or amino acid sequences, refers to two or more sequences or subsequences that are the same or that have a specified percentage of amino acid residues or nucleotides that are the same (e.g., at least 65% identity, preferably, at least 70-95% identity, more preferably at least 95%, 96%, 97%, 98% or 99% identity), when compared and aligned for maximum correspondence over a window of comparison or over a designated region as measured using a sequence comparison algorithm as known in the art, or by manual alignment and visual inspection. Sequences having, for example, 65% to 95% or greater sequence identity are considered to be substantially identical. Such a definition also applies to the complement of a test sequence. Preferably the described identity exists over a region that is at least about 232 amino acids or 696 nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3 and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands.

For example, BLAST2.0, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence to evaluate the statistical significance of any matches found and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules.

Most preferably, the methyl-DNA-binding domain or fragment or variant thereof of a polypeptide of the present invention employed in the method of the present invention comprises the methyl-DNA-binding domain of the MBD proteins shown in FIG. 1 of Ballester and Wolffe (2001), loc. cit. or the methyl-DNA-binding domain of the MBD proteins described in Hendrich and Tweedy, Trends Genet. 19 (2003), 269-77 and is able to bind methylated DNA.

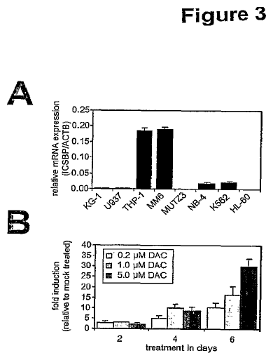

In a particular preferred embodiment of the invention, the methyl-DNA-binding domain of a polypeptide employed in the method of the present invention is that of human MBD2. In a more particular preferred embodiment, the methyl-DNA-binding domain is that of human MBD2 comprising amino acids 144 to 230 of the amino acid sequence having Genbank accession number NM 003927. In a most particular preferred embodiment, the methyl-DNA-binding domain of a polypeptide employed in the method of the present invention comprises the amino acid sequence from position 29 to 115 of the amino acid sequence shown in SEQ ID NO:2 (FIG. 3).

A “variant” of a polypeptide of the present invention which is capable of binding methylated DNA and which is employed in the method of the present invention encompasses a polypeptide wherein one or more amino acid residues are substituted, preferably conservatively substituted compared to said polypeptide and wherein said variant is preferably able to bind to methylated DNA, preferably CpG methylated DNA. Such variants include deletions, insertions, inversions, repeats, and substitutions selected according to general rules known in the art, so as have no effect on the activity of a polypeptide of the present invention. For example, guidance concerning how to make phenotypically silent amino acid substitutions is provided in Bowie, Science 247: (1990) 1306-1310, wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicate that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244: (1989) 1081-1085.) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved.

The invention encompasses polypeptides having a lower degree of identity but having sufficient similarity, so as to perform one or more of the functions performed by a polypeptide as described herein which is employed in the method of the present invention. Similarity is determined by conserved amino acid substitution. Such substitutions are those that substitute a given amino acid in a polypeptide by another amino acid of like characteristics (e.g., chemical properties). According to Cunningham et al. above, such conservative substitutions are likely to be phenotypically silent. Additional guidance concerning which amino acid changes are likely to be phenotypically silent are found in Bowie, Science 247: (1990) 1306-1310.

Tolerated conservative amino acid substitutions of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In addition, the present invention also encompasses the conservative substitutions provided in the Table 2 below.

TABLE 2

| For Amino Acid | Code | Replace with any of: |
|---|---|---|
| Alanine | A | D-Ala, Gly, beta-Ala, L-Cys, D-C_{y}s |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-As |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, D- |
| | | or L-1-oxazolidine-4-carboxylic acid |
| Serine | S | D-Ser, Thr, D-Thr, allo-Thr, Met, D-Met, |
| | | Met(O), D-Met(0), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Aside from the uses described above, such amino acid substitutions may also increase protein or peptide stability. The invention encompasses amino acid substitutions that contain, for example, one or more non-peptide bonds (which replace the peptide bonds) in the protein or peptide sequence. Also included are substitutions that include amino acid residues other than naturally occurring L-amino acids, e.g., D-amino acids or non-naturally occurring or synthetic amino acids, e.g., B or y amino acids.

Both identity and similarity can be readily calculated by reference to the following publications: Computational Molecular Biology, Lesk, A. M., ed., Oxford University Press, New York, 1988; Biocomputing: Infolivaties and Genome Projects, Smith, DM., ed., Academic Press, New York, 1993; Informafies Computer Analysis of Sequence Data, Part 1, Griffin, A. M., and Griffin, H. G., eds., Humana Press, New Jersey, 1994; Sequence Analysis in Molecular Biology, von Heinje, G., Academie Press, 1987; and Sequence Analysis Primer, Gribskov, M. and Devereux, eds., M Stockton Press, New York, 1991.

As mentioned herein above, a polypeptide to be used for binding methylated DNA also encompasses preferably an anti-methylated DNA antibody which is preferably an anti-5-methylcytosine antibody or a Fab, F(ab′)₂, Fv or scFv fragment thereof. Preferably, said anti-5-methylcytosine antibody specifically binds to methylated DNA, preferably CpG-methylated DNA. The term “specifically” in this context means that said antibody reacts with CpG-methylated DNA, but not with unmethylated DNA and/or DNA methylated at other nucleotides than cytosine and/or DNA methylated at other positions than the C5 atom of cytosine.

Whether the antibody specifically reacts as defined herein above can easily be tested, inter alia, by comparing the binding reaction of said antibody with CpG-methylated DNA and with unmethylated DNA and/or DNA methylated at other nucleotides than cytosine and/or DNA methylated at other positions than the C5 atom of cytosine.

The antibody of the present invention can be, for example, polyclonal or monoclonal. The term “antibody” also comprises derivatives or fragments thereof which still retain the binding specificity such as a Fab, F(ab′)₂, Fv, or scFv fragment. Techniques for the production of antibodies are well known in the art and described, e.g. in Harlow and Lane “Antibodies, A Laboratory Manual”, CSH Press, Cold Spring Harbor, 1988. The present invention furthermore includes chimeric, single chain, and humanized antibodies, as well as antibody fragments as mentioned above; see also, for example, Harlow and Lane, loc. cit.. Various procedures are known in the art and may be used for the production of such antibodies and/or fragments. Thus, the (antibody) derivatives can be produced by peptidomimetics. Further, techniques described for the production of single chain antibodies (see, inter alia, U.S. Pat. No. 4,946,778) can be adapted to produce single chain antibodies to polypeptide(s) of this invention. Also, transgenic animals may be used to express humanized antibodies to polypeptides of this invention. Most preferably, the anti-methylated DNA antibody of this invention is a monoclonal antibody. For the preparation of monoclonal antibodies, any technique which provides antibodies produced by continuous cell line cultures can be used. Examples for such techniques include the hybridoma technique (Kohler and Milstein Nature 256 (1975), 495-497), the trioma technique, the human B-cell hybridoma technique (Kozbor, Immunology Today 4 (1983), 72), and the EBV-hybridoma technique to produce human monoclonal antibodies (Cole et al., Monoclonal Antibodies and Cancer Therapy, Alan R. Liss, Inc. (1985), 77-96). Techniques describing the production of single chain antibodies (e.g., U.S. Pat. No. 4,946,778) can be adapted to produce single chain antibodies to immunogenic polypeptides as described above. Accordingly, in context of the present invention, the term “antibody molecule” relates to full immunoglobulin molecules as well as to parts of such immunoglobulin molecules. Furthermore, the term relates, as discussed above, to modified and/or altered antibody molecules, like chimeric and humanized antibodies. The term also relates to monoclonal or polyclonal antibodies as well as to recombinantly or synthetically generated/synthesized antibodies. The term also relates to intact antibodies, as well as to antibody fragments thereof, like separated light and heavy chains, Fab, Fab/c, Fv, Fab′, F(ab′)2. The term “antibody molecule” also comprises bifunctional antibodies and antibody constructs like single chain Fvs (scFv) or antibody-fusion proteins. It is also envisaged in context of this invention that the term “antibody” comprises antibody constructs which may be expressed in cells, e.g. antibody constructs which may be transfected and/or transduced via, inter alia, viruses or vectors. Of course, the antibody of the present invention can be coupled, linked, or conjugated to detectable substances.

Examples of detectable substances include various enzymes, prosthetic groups, fluorescent materials, luminescent materials, bioluminescent materials, radioactive materials, positron emitting metals using various positron emission tomographies and nonradioactive paramagnetic metal ions. The detectable substance may be coupled or conjugated either directly to an Fc portion of an antibody (or fragment thereof) or indirectly, through an intermediate (such as, for example, a linker known in the art) using techniques known in the art. See, for example, U.S. Pat. No. 4,741,900 for metal ions which can be conjugated to an Fc portion of antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; examples of bioluminescent materials include luciferase, luciferin, and aequorin; and examples of suitable radioactive material include ¹²⁵I, ¹³¹I, or ⁹⁹Tc.

Further, said Fc portion may be conjugated to a therapeutic moiety such as a cytotoxin, e.g., a cytostatic or cytocidal agent, a therapeutic agent or a radioactive metal ion, e.g., alpha-emitters such as, for example, ²¹³Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, analogs, or homologues thereof. Therapeutic agents include, but are not limited to, antimetabolites (e.g., methotrexate, 6-mereaptopurine, 6-thioguanine, cytarabine, 5-fluorouracil decarbazine), alkylating agents (e.g., mechlorethamine, thioepa chlormbucil, melphalan, carmustine (BSNU) and lomustine (CCNU), cyclothosphamide, busulfan, dibromomannitol, streptozotocin, mitomycin C, and cisdichlorodiamine platinum (11) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Furthermore, the Fc portion of the polypeptide of the present invention may be coupled or conjugated to a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator, or an apoptotic agent.

The Fc portion also allows attachment of the polypeptide of the present invention to solid supports, which are particularly useful for immunoassays or purification of the target antigen as described herein. Such solid supports include, but are not limited to, glass, cellulose, polyacrylamide, nylon, polycabonate, polystyrene, polyvinyl chloride or polypropylene or the like.

Techniques for conjugating coupling or linked compounds to the Fc portion are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies ‘84: Biological And Clinical Applications, Pinchera et al. (eds.), pp. 475-506 (1985); “Analyzis, Results, And Future Prospective Of The Therapeutic Use Of Radiolabeled Antibody In Cancer Therapy”, in Monoelonal Antibodies For Cancer Detection And Therapy, Baldwin et al. (eds.), pp. 303-16 (Academic Press 1985), and Thorpe, Immunol. Rev., 119-158.

In a preferred embodiment of the present invention, a polypeptide as described herein which is used in the method of the present invention is fused at its N- and/or C-terminus to a heterologous polypeptide for detecting methylated DNA which is preferably selected from the group consisting of a HA-tag, myc6-tag, FLAG-tag, STREP-tag, STREP II-tag, TAP-tag, HAT-tag, chitin-binding domain (CBD), maltose-binding protein, His6-tag, Glutathione-S-transferase (GST) tag, Intein-tag, Streptavidin-binding protein (SBP) tag, and a Fc-portion of an antibody. A “tag” is an amino acid sequence which is homologous or heterologous to an amino acid sequence to which it is fused. Said tag may, inter alia, facilitate purification of a protein or facilitate detection of said protein to which it is fused. The fusion refers to a co-linear linkage and results in a translation fusion. In an also further preferred embodiment a polypeptide of the present invention which is capable of binding methylated DNA is fused to a heterologous polypeptide and optionally comprises an additional linker between the N- and/or C-terminus of said polypeptide and said heterologous polypeptide. Said linker is preferably a flexible linker. Preferably, it comprises plural, hydrophilic peptide-bonded amino acids. Optionally, the linker comprises a protease cleavage site which allows to cut off the heterologous polypeptide fused to a polypeptide of the present invention, if desirable. Protease cleavage sites are, for example, a thrombin cleavage site.

Preferably, said linker comprises a plurality of glycine, alanine, aspartate, glutamate, proline, isoleucine, and/or arginine residues. It is further preferred that said polypeptide linker comprises a plurality of consecutive copies of an amino acid sequence. Usually, the polypeptide linker comprises 1 to 20, preferably 1 to 19, 1 to 18, 1 to 17, 1 to 16, or 1 to 15 amino acids although polypeptide linkers of more than 20 amino acids may work as well.

Preferably, said Fc protein of an antibody comprises preferably at least a portion of the constant region of an immunoglobulin heavy chain molecule. The Fc region is preferably limited to the constant domain hinge region and the C_{H}2 and C_{H}3 domains. The Fc region in a polypeptide of the present invention which is capable of binding methylated DNA and which is employed in the method of the present invention can also be limited to a portion of the hinge region, the portion being capable of forming intermolecular disulfide bridges, and the C_{H}2 and C_{H}3 domains, or functional equivalents thereof.

Alternatively, it is also preferred that the Fc portion comprises at least so many CH regions which are required such that a polypeptide of the present invention capable of binding methylated DNA has still the properties of a polypeptide described hereinabove, in particular the properties of the polypeptide used in the appended Examples.

In another alternative, it is also preferred that said constant region may contain one or more amino acid substitutions when compared to constant regions known in the art. Preferably it contains 1 to 100, 1 to 90, 1 to 80, 1 to 70, 1 to 60, 1 to 50, 1 to 40, 1 to 30, or 1 to 20, more preferably 1 to 10, even more preferably 1 to 9, 1 to 8, 1 to 7 or 1 to 6, and most preferably 1 to 5, 1 to 4, 1 to 3 or 2 or 1 substitution(s). The comparison is preferably done as is known in the art or, more preferably, as described elsewhere herein.

Alternatively, said constant region comprises preferably at least the C_{H}1 region, more preferably the C_{H}1 and C_{H}2 regions, and most preferably the C_{H}1, C_{H}2 and C_{H}3 region. As is known in the art, the constant region of an antibody contains two immunoglobulin heavy chains which harbour three characteristic immunoglobulin domains composed of about 110 amino acids, wherein the two immunoglobulin heavy chains are covalently linked via disulfide bonds.

It is also envisaged that the constant region could preferably be of chicken or duck origin. Yet, preferably, the constant region is of the IgM, IgA, IgD or IgE isotype and more preferably it is of the IgG isotype, most preferably of the IgG1 isotype. Preferably, the aforementioned isotypes are of vertebrate origin, more preferably of mammal origin, even more preferably of mouse, rat, goat, horse, donkey, camel or chimpanzee origin and most preferably of human origin. Preferably, said IgG isotype is of class IgG1, IgG2, IgG3, IgG4, and said IgA isotype is of class IgA1 or IgA2.

As described herein, the present invention provides preferably for bifunctional polypeptides. Yet, also multimeric bifunctional polypeptides comprising one or more of the bifunctional polypeptide of the present invention are envisaged. Such multimers may be generated by using those Fc regions, or portions thereof, of Ig molecules which are usually multivalent, such as IgM pentamers or IgA dimers. It is understood that a J chain polypeptide may be needed to form and stabilize IgM pentamers and IgA dimers.

In a further preferred embodiment of the present invention a polypeptide used in the method of the present invention is a fusion protein between the methyl-DNA binding domain of the MBD2 protein and the Fc portion of an antibody as disclosed herein. Optionally the preferred fusion protein comprises a linker polypeptide as described herein, wherein said linker polypeptide is preferably located between the methyl-DNA binding domain of MBD2 and the Fc portion of an antibody.

The herein described heterologous polypeptide fused to a polypeptide used in the method of the present invention facilitates binding and/or attachment of a polypeptide used in the method of the present inventions to a container or solid support including, but not limited to, glass, cellulose, polyacrylamide, nylon, polycarbonate, polystyrene, polyvinyl chloride or polypropylene or the like. Preferably, said container is a PCR-tube composed of polycarbonate, and more preferably, it is a heat stable TopYield™ strip from Nunc Cat. No. 248909. Said PCR-tube or strip may be in the format of a 96-well, 384-well or 1024-well plate. Accordingly, the method of the present invention is suitable for high-through put applications which can be automated since the method of the present invention can be performed as so-called “one tube—one assay”.

In a preferred embodiment, the container or solid support, preferably a PCR-tube or stripe, is coated directly or indirectly with a polypeptide used in the method of the present invention: for example, coating would be achieved directly by using a biotinylated polypeptide of the present invention and a streptavidin coated container, preferably a PCR-tube. However, any other technique known in the art for coating a container with a polypeptide are contemplated by the present invention. Indirect coating can preferably be achieved by an antibody coated onto the surface of said container and which is capable to specifically bind either a polypeptide of the present invention which is capable of binding methylated DNA or specifically binding the heterologous polypeptide preferably fused to said polypeptide capable of binding methylated DNA or specifically binding the anti-methylated DNA antibody of the present invention. In fact, said container is indirectly coated with a polypeptide of the present invention which is capable of binding methylated DNA.

Coating of the container as described herein may be achieved, for example, by coating said container with an agent which is suitable to interact with the heterologous polypeptide fused to a polypeptide of the present invention which is capable of binding methylated DNA. For example, said container may be coated with glutathione and, accordingly, a GST-tagged polypeptide of the present invention is bound by glutathione which results in coating of said container with a polypeptide to be employed in the method of the present invention. Preferably, coating of the container occurs due to the property of the plastic out of which the preferred container described herein is built. Accordingly, when a polypeptide of the present invention is brought in contact with a container of the present invention, said polypeptide coats said container.

As mentioned herein, the method of the present invention allows the detection of methylated DNA of, preferably, a single gene locus which renders it a suitable diagnostic tool for, inter alia, detecting methylated DNA from more than 15 μg, less than 15 μg, less than 10 μg, less than 10 ng, 7.5 ng, 5 ng, 2.5 ng, 1 ng, 0.5 ng, 0.25 ng, or about 150 pg. By the term biological sample obtained from a subject or an individual, cell line, tissue culture, or other source containing polynucleotides, polypeptides, or portions thereof. As indicated, biological samples include body fluids (such as blood, sera, plasma, urine, synovial fluid, and spinal fluid) and tissue sources found to express the polynucleotides of the present invention. Methods for obtaining tissue biopsies and body fluids from mammals are well known in the art. A biological sample which includes genomic DNA, mRNA, or proteins is preferred as a source.

Without being bound by theory, it is believed that methylation of CpG dinucleotides correlates with stable transcriptional repression and presumably leads to the fact that large parts of the non-coding genome and potentially harmful sequences are not transcribed. A global DNA hypomethylation has been described for almost all kinds of tumors. In tumor tissue, the content in 5-methylcytosine is reduced compared to normal tissue with the major share of demethylation events being found in repetitive satellite sequences or in centromer regions of the chromosomes. However, in single cases, the demethylation and activation of proto-oncogenes such as, e.g., bcl-2 or c-myc have also been described (Costello, J. Med. Genet. 38 (2001), 285-303).

CpG islands in general exert gene regulatory functions. This is why a change in the status of methylation correlates mostly directly with a change in the transcriptional activity of the locus concerned (Robertson (1999); Herman (2003); Esteller (2002); Momparler (2003); Plass (2002), all loc. cit.). Most CpG islands are present in unmethylated form in normal cells. In certain situations, CpG islands can, however, also be methylated in gene regulatory events. The majority of CpG islands of the inactivated X-chromosome of a female cell are, for example, methylated (Goto, Microbiol. Mol. Biol. Rev. 62 (1998), 362-378). CpG islands can be methylated also in the course of normal aging processes (Issa, Clin. Immunol. 109 (2003), 103-108).

It is in particular in tumors that CpG islands which are normally not methylated can be present in a hypermethylated form. In many cases, genes affected by the hypermethylation encode proteins which counteract the growth of a tumor such as, e.g., tumor suppressor genes. Examples of genes for which it could be shown that they can be inactivated in tumors through the epigenetic mechanism of hypermethylation are described herein above. Reasons for the tumor-specific hypermethylation are almost unknown. Interestingly, certain kinds of tumors seem to have their own hypermethylation profiles. It could be shown in larger comparative studies that hypermethylation is not evenly distributed, but that it occurs depending on the tumor. In cases of leukaemia, mostly other genes are hypermethylated compared to, for instance, colon carcinomas or gliomas. Thus, hypermethylation could be useful for classifying tumors (Esteller, Cancer Res. 61 (2001), 3225-3229; Costello, Nat. Genet. 24 (2000), 132-138).

Thus, it is believed that epigenetic effects such as hypo and/or hypermethylation are correlated with cancers, tumors, and/or metastatis.

The subject of the present invention from which the sample is obtained for detecting methylated DNA is suspected to have hypo- and/or hypermethylated genloci. Said hypo and/or hypermethylated genloci are indicative of a cancer, tumor or metastasis. The tumor or cancer can be any possible type of tumor or cancer. Examples are skin, breast, brain, cervical carcinomas, testicular carcinomas, head and neck, lung, mediastinum, gastrointestinal tract, genitourinary system, gynaecological system, breast, endocrine system, skin, childhood, unknown primary site or metastatic cancer, a sarcoma of the soft tissue and bone, a mesothelioma, a melanoma, a neoplasm of the central nervous system, a lymphoma, a leukaemia, a paraneoplastic syndrome, a peritoneal carcinomastosis, a immunosuppression-related malignancy, and/or metastatic cancer etc. The tumor cells may, e.g., be derived from: head and neck, comprising tumors of the nasal cavity, paranasal sinuses, nasopharynx, oral cavity, oropharynx, larynx, hypopharynx, salivary glands, and paragangliomas, a cancer of the lung, comprising non-small cell lung cancer, small cell lung cancer, a cancer of the mediastinum, a cancer of the gastrointestinal tract, comprising cancer of the oesophagus, stomach, pancreas, liver, biliary tree, small intestine, colon, rectum and anal region, a cancer of the genitourinary system, comprising cancer of the kidney, urethra, bladder, prostate, urethra, penis and testis, a gynaecologic cancer, comprising cancer of the cervix, vagina, vulva, uterine body, gestational trophoblastic diseases, ovarian, fallopian tube, peritoneal, a cancer of the breast, a cancer of the endocrine system, comprising a tumor of the thyroid, parathyroid, adrenal cortex, pancreatic endocrine tumors, carcinoid tumor and carcinoid syndrome, multiple endocrine neoplasias, a sarcoma of the soft tissue and bone, a mesothelioma, a cancer of the skin, a melanoma, comprising cutaneous melanomas and intraocular melanomas, a neoplasm of the central nervous system, a cancer of the childhood, comprising retinoblastoma, Wilm's tumor, neurofibromatoses, neuroblastoma, Ewing's sarcoma family of tumors, rhabdomyosarcoma, a lymphoma, comprising non-Hodgkin's lymphomas, cutaneous T-cell lymphomas, primary central nervous system lymphoma, and Hodgkin's disease, a leukaemia, comprising acute leukemias, chronic myelogenous and lymphocytic leukemias, plasma cell neoplasms and myelodysplastic syndromes, a paraneoplastic syndrome, a cancer of unknown primary site, a peritoneal carcinomastosis, a immunosuppression-related malignancy, comprising AIDS-related malignancies, comprising Kaposi's sarcoma, AIDS-associated lymphomas, AIDS-associated primary central nervous system lymphoma, AIDS-associated Hodgkin's disease and AIDS-associated anogenital cancers, and transplantation-related malignancies, a metastatic cancer to the liver, metastatic cancer to the bone, malignant pleural and pericardial effusions and malignant ascites. It is mostly preferred that said cancer or tumorous disease is cancer of the head and neck, lung, mediastinum, gastrointestinal tract, genitourinary system, gynaecological system, breast, endocrine system, skin, childhood, unknown primary site or metastatic cancer, a sarcoma of the soft tissue and bone, a mesothelioma, a melanoma, a neoplasm of the central nervous system, a lymphoma, a leukaemia, a paraneoplastic syndrome, a peritoneal carcinomastosis, a immunosuppression-related malignancy and/or metastatic cancer. Preferred tumors are AML, plasmacytoma, or CLL.

As mentioned herein, the present invention provides a method for detecting methylated DNA, preferably CpG-methylated DNA fragments in a single-tube assay comprising the following steps: binding of genomic DNA to polypeptide which is capable of binding methylated DNA, preferably a methyl-CpG-binding protein, coated onto to the inner surface of a container, preferably a PCR-tube, washing off unbound (unmethylated) DNA-fragments and preferably directly applying gene-specific PCR to detect the enrichment of methylated DNA. Since the method of the present invention is robust, fast and is an easy applicable and reliable diagnostic tool for detecting methylated DNA due to the “one reaction container for all steps,” the method of the present invention may be applicable to high through put formats which may be made subject of automation. The method of the present invention allows thus an easy and highly sensitive detection of CpG methylation of preferably (a) single gene locus/loci. Since methylation patterns of tumors and/or cancers appear to develop into a valuable diagnostic parameter, it is preferred to provide a kit comprising all means for carrying out the method of the present invention.

Accordingly, the present invention relates to a kit comprising for detecting methylated DNA according to the method of the present invention comprising
(a) a polypeptide capable of binding methylated DNA as described herein;
(b) a container which can be coated with said polypeptide; and
(c) means for coating said container; and
(d) means for detecting methylated DNA.

The embodiments disclosed in connection with the method of the present invention apply, mutatis mutandis, to the kit of the present invention.

Advantageously, the kit of the present invention further comprises, optionally (a) reaction buffer(s), storage solutions, wash solutions and/or remaining reagents, or materials required for the conduction of scientific or diagnostic assays or the like, as described herein. Furthermore, parts of the kit of the invention can be packaged individually in vials or bottles or in combination in containers or multi-container units.

The kit of the present invention may be advantageously used, inter alia, for carrying out the method for detecting methylated DNA as described herein, and/or it could be employed in a variety of applications referred herein, e.g., as diagnostic kits, as research tools or therapeutic tools. Additionally, the kit of the invention may contain means for detection suitable for scientific, medical, and/or diagnostic purposes. The manufacture of the kits follows preferably standard procedures which are known to the person skilled in the art. The kit of the present invention is preferably useful in a “single-tube” assay as provided herein.

“Means for coating” of the container of the present invention are all agents suitable for coating said container with a polypeptide of the present invention, for example, cross-linking agents, avidin, glutathione, or the like. Thus, basically, every agent which is suitable to interact with the heterologous polypeptide fused to a polypeptide of the present invention which is capable of binding methylated DNA. Preferably, the kit of the present invention comprises pre-coated containers, preferably PCR-tubes.

The term “means for detecting methylated DNA” encompasses all agents necessary to carry out the detection methods for methylated DNA as described herein above. In a more preferred embodiment, said kit comprises an instruction manual how to carry out detection of methylated DNA according to the method of the present invention.

The present invention provides for diagnostic composition comprising at least one of the herein described compounds of the invention. The diagnostic composition may be used, inter alia, for methods for isolating, enriching, and/or determining the presence of methylated

DNA, preferably CpG methylated DNA, for example, in a sample from an individual as described above.

Further applications of the diagnostic compositions are described herein and are shown in the appended Examples.

The diagnostic composition optionally comprises suitable means for detection. The nucleic acid molecule(s), vector(s), host(s), antibody(ies), and polypeptide(s) described above are, for example, suitable for use in immunoassays in which they can be utilized in liquid phase or bound to a solid phase carrier. Examples of well-known carriers include glass, polystyrene, polyvinyl ion, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

Solid phase carriers are known to those in the art and may comprise polystyrene beads, latex beads, magnetic beads, colloid metal particles, glass and/or silicon chips and surfaces, nitrocellulose strips, membranes, sheets, duracytes and the walls of wells of a reaction tray, plastic tubes or other test tubes. Suitable methods of immobilizing nucleic acid molecule(s), vector(s), host(s), antibody(ies), aptamer(s), polypeptide(s), etc. on solid phases include but are not limited to ionic, hydrophobic, covalent interactions or (chemical) crosslinking, and the like. Examples of immunoassays which can utilize said compounds of the invention are competitive and non-competitive immunoassays in either a direct or indirect format. Commonly used detection assays can comprise radioisotopic or non-radioisotopic methods. Examples of such immunoassays are the radioimmunoassay (RIA), the sandwich (immunometric assay) and the Northern or Southern blot assay. Furthermore, these detection methods comprise, inter alia, IRMA (Immune Radioimmunometric Assay), EIA (Enzyme Immuno Assay), ELISA (Enzyme Linked Immuno Assay), FIA (Fluorescent Immuno Assay), and CLIA (Chemioluminescent Immune Assay). Furthermore, the diagnostic compounds of the present invention may be are employed in techniques like FRET (Fluorescence Resonance Energy Transfer) assays.

Appropriate labels and methods for labeling are known to those of ordinary skill in the art. Examples of the types of labels which can be used in the present invention include inter alia, fluorochromes (like fluorescein, rhodamine, Texas Red, etc.), enzymes (like horse radish peroxidase, β-galactosidase, alkaline phosphatase), radioactive isotopes (like ³²P, ³³P, ³⁵S or ¹²⁵I), biotin, digoxygenin, colloidal metals, chemi- or bioluminescent compounds (like dioxetanes, luminol, or acridiniums).

A variety of techniques are available for labeling biomolecules, are well known to the person skilled in the art, and are considered to be within the scope of the present invention and comprise, inter alia, covalent coupling of enzymes or biotinyl groups, phosphorylations, biotinylations, random priming, nick-translations, and tailing (using terminal transferases). Such techniques are, e.g., described in Tijssen, “Practice and theory of enzyme immunoassays”, Burden and von Knippenburg (Eds), Volume 15 (1985); “Basic methods in molecular biology”, Davis L G, Dibmer M D, Battey Elsevier (1990); Mayer, (Eds) “Immunochemical methods in cell and molecular biology” Academic Press, London (1987); or in the series “Methods in Enzymology”, Academic Press, Inc. Detection methods comprise, but are not limited to, autoradiography, fluorescence microscopy, direct and indirect enzymatic reactions, etc.

Another preferred composition of the present invention is a pharmaceutical composition optionally further comprising a pharmaceutical acceptable carrier. Said pharmaceutical composition comprises, inter alia, the polypeptide of the present invention which may be coupled to a further polypeptide, for example, a histone deacetylase, a histone acetylase, DNA-methylase, and/or DNA-demethylase. It could also be coupled with a restriction enzyme or a ribozyme. It is believed that if the polypeptide of the present invention coupled with one or more further protein, as described above, binds to methylated DNA, it may target said further protein(s) to DNA. Accordingly, a DNA-methylase could hyper-methylate a hypomethylated DNA, for example, a hypomethylated oncogenic locus or oncogene or a DNA. In doing so, gene inactivation could be achieved.

Alternatively, a DNA-demethylase may demethylate a hypermethylated gene or genlocus, for example, a tumor suppressor gene or genlocus. In doing so, gene activation could be achieved.

A histone deacetylase contribute to transcriptional repression of an active gene by deacetylating acetylated lysine residues of histones, thereby leading to a tighter packaging of DNA to histones and, gene repression. A histone acetylase could do the contrary effect as is known in the art.

A restriction enzyme or a ribozyme could exert its effect when targeted to DNA which should be cleaved. Appropriate restriction enzymes are known in the art. Ribozymes specific for target-DNA sequences can be prepared as is known in the art.

Accordingly, the pharmaceutical composition could be useful for treating cancer and/or tumorous disease. Both of which are known to be caused by uncontrolled gene expression, activation and/or repression which is, inter alia, regulated by histone acetylation/deacetylation and/or DNA-methylation/demethylation.

The pharmaceutical composition may be administered with a physiologically acceptable carrier to a patient, as described herein. In a specific embodiment, the term “pharmaceutically acceptable” means approved by a regulatory agency or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, and the like. Water is a preferred carrier when the pharmaceutical composition is administered intravenously. Saline solutions, aqueous dextrose, and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium ion, dried skim milk, glycerol, propylene, glycol, water, ethanol and the like. The composition, if desired, can also contain minor amounts of wetting or emulsifying agents or pH buffering agents. These compositions can take the form of solutions, suspensions, emulsion, tablets, pills, capsules, powders, sustained-release formulations, and the like. The composition can be formulated as a suppository with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the aforementioned compounds, preferably in purified form, together with a suitable amount of carrier so as to provide the form for proper administration to the patient. The formulation should suit the mode of administration.

In another preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical composition of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In vitro assays may optionally be employed to help identify optimal dosage ranges. The precise dose to be employed in the formulation will also depend on the route of administration and the seriousness of the disease or disorder, and it should be decided according to the judgment of the practitioner and each patient's circumstances. Effective doses may be extrapolated from dose-response curves derived from in vitro or animal model test systems. Preferably, the pharmaceutical composition is administered directly or in combination with an adjuvant.

The pharmaceutical composition is preferably designed for the application in gene therapy. The technique of gene therapy has already been described above in connection with the nucleic acid molecules of the invention and all what has been said there also applies in connection with the pharmaceutical composition. For example, the nucleic acid molecule in the pharmaceutical composition is preferably in a form which allows its introduction, expression and/or stable integration into cells of an individual to be treated.

For gene therapy, various viral vectors which can be utilized, for example, adenovirus, herpes virus, vaccinia, or, preferably, an RNA virus such as a retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can also incorporate multiple genes. All of these vectors can transfer or incorporate a gene for a selectable marker so that transduced cells can be identified and generated. Retroviral vectors can be made target specific by inserting, for example, a polynucleotide encoding a sugar, a glycolipid, or a protein. Those of skill in the art will know of, or can readily ascertain without undue experimentation, specific polynucleotide sequences which can be inserted into the retroviral genome to allow target specific delivery of the retroviral vector containing the inserted polynucleotide sequence.

Since recombinant retroviruses are preferably defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsidation. Helper cell lines which have deletions of the packaging signal include, but are not limited to w2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced. Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium. Another targeted delivery system for the nucleic acid molecules of the present invention is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 pm can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast, and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine, and distearoylphosphatidylcholine. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

In a preferred embodiment, the compositions of the present invention may be useful for in vivo imaging methylated DNA, preferably CpG methylated DNA. Accordingly said composition is administered to a subject in need thereof. In the context of the present invention the term “subject” means an individual in need of a treatment of an affective disorder. Preferably, the subject is a vertebrate, even more preferred a mammal, particularly preferred a human. The term “administered” means administration of a therapeutically or diagnostically effective dose of the aforementioned nucleic acid molecule encoding the polypeptide of the present invention to an individual. By “therapeutically or diagnostically effective amount” is meant a dose that produces the effects for which it is administered. The exact dose will depend on the purpose of the treatment or diagnosis and will be ascertainable by one skilled in the art using known techniques. As is known in the art and described above, adjustments for systemic versus localized delivery, age, body weight, general health, sex, diet, time of administration, drug interaction, and the severity of the condition may be necessary and will be ascertainable with routine experimentation by those skilled in the art. The methods are applicable to both human therapy and veterinary applications. The compounds described herein having the desired therapeutic activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-100 wt %. The agents may be administered alone or in combination with other treatments.

The administration of the pharmaceutical composition can be done in a variety of ways as discussed above, including, but not limited to, orally, subcutaneously, intravenously, intra-arterial, intranodal, intramedullary, intrathecal, intraventricular, intranasally, intrabronchial, transdermally, intranodally, intrarectally, intraperitoneally, intramuscularly, intrapulmonary, vaginally, rectally, or intraocularly. In some instances, for example, in the treatment of wounds and inflammation, the candidate agents may be directly applied as a solution dry spray.

The attending physician and clinical factors will determine the dosage regimen. As is well known in the medical arts, dosages for any one patient depends upon many factors, including the patient's size, body surface area, age, the particular compound to be administered, sex, time, and route of administration, general health, and other drugs being administered concurrently. A typical dose can be, for example, in the range of 0.001 to 1000 μg; however, doses below or above this exemplary range are envisioned, especially considering the aforementioned factors.

The dosages are preferably given once a week, however, during progression of the treatment the dosages can be given in much longer time intervals and, in need, can be given in much shorter time intervals, e.g., daily. In a preferred case, the immune response is monitored using herein described methods and further methods known to those skilled in the art and dosages are optimized, e.g., in time, amount and/or composition. Dosages will vary but a preferred dosage for intravenous administration of DNA is from approximately 10⁶ to 10¹² copies of the DNA molecule. If the regimen is a continuous infusion, it should also be in the range of 1 μg to 10 mg units per kilogram of body weight per minute, respectively. Progress can be monitored by periodic assessment. The pharmaceutical composition of the invention may be administered locally or systemically. Administration will preferably be parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium ion solution, Ringer's dextrose, dextrose and sodium ion, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

It is also envisaged that the pharmaceutical compositions are employed in co-therapy approaches with other agents are, for example, useful in detecting methylated DNA and, thus, for example, useful in diagnosing malignancies which may show a typical methylated pattern.

The figures show:

FIG. 1: Outline of Methyl-binding (MB)-PCR. (A) The major steps of the MB-PCR procedure are illustrated. MB-PCR comprises of two separate reactions, the control-PCR reaction (P-reaction) which amplifies a candidate locus directly from a genomic template, and the methyl-CpG-binding-PCR reaction which amplifies the candidate locus from the template DNA that was previously bound by a methyl-CpG-binding polypeptide in the reaction vessel (M-reaction). In the first step, the inner walls of both reaction vessels are coated with a methyl-binding polypeptide and subsequently saturated using blocking reagents (step 2). The template DNA (genomic DNA restricted with Mse I or similar enzymes) is then added to one tube (M-reaction) and allowed to bind (step 3). In the last step, the PCR reaction mix is added directly into both tubes and 50% of template DNA previously used for the M-reaction is added to the P-reaction. After gene-specific PCR, products may be analyzed, e.g. by agarose gel electrophoresis. The term “CpG-methylation low” used in FIGS. 1 A and B comprises and particularly refers to unmethylated DNA (B) Schematic representation of the MB-PCR procedure using a recombinant methyl-binding polypeptide MBD-Fc described herein above.

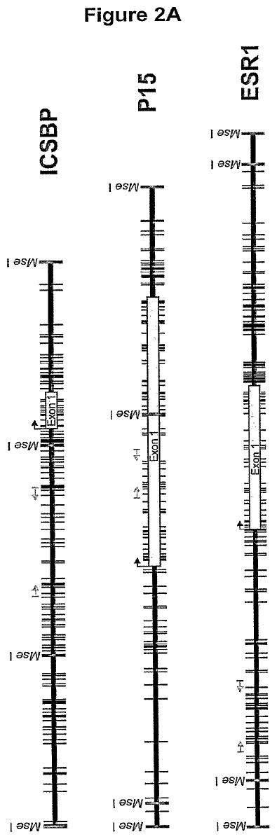

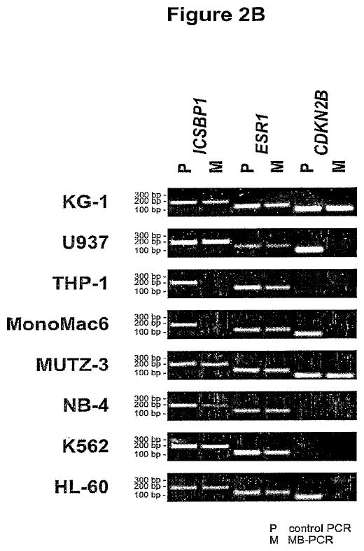

FIG. 2: Detecting CpG methylation in leukaemia cell lines at three CpG-island promoters by MB-PCR. (A) Shown are: the position of CpG-dinucleotides, Mse I-restriction sites, first exons and positions of primers used to detect promoter fragments of ICSBP, ESR1, and CDKN2B (p15^{INK4b}). (B) Representative MB-PCR results of the indicated promoters for eight different leukaemia cell lines. The P-reaction directly amplifies the genomic DNA, whereas the M-reaction only amplifies CpG-methylated DNA fragments.

FIG. 3: Methylation of the ICSBP promoter inversely correlates with ICSBP expression in leukaemia cell lines. (A) Transcription levels of ICSBP were determined by LightCycler real time PCR relative to the housekeeping gene ACTB. (B) U937 cells, treated with Decitabine (DAC) for the indicated time periods were analyzed for ICSBP expression. Results were normalized to ACTB expression. Data represent mean values±SD of two independent LightCycler analyzes.

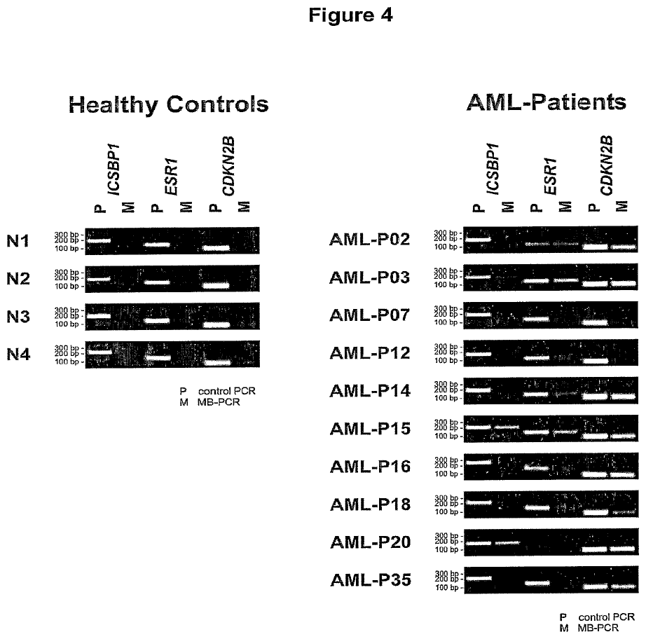

FIG. 4: Detection of aberrant CpG methylation in AML cells. Representative MB-PCRs for ESR1, CDKN2B (p15^{INK4b}), and ICSBP promoters of several healthy donors and AML patients.

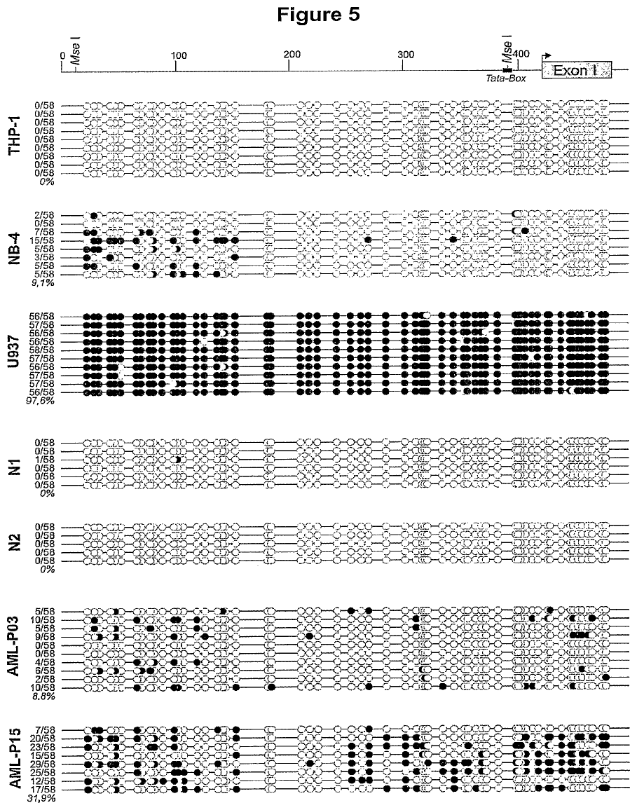

FIG. 5: MB-PCR of the ICSBP promoter correlates with the results obtained by bisulfite sequencing. Genomic DNA derived from cell lines as well as cells of selected healthy donors and AML patients was treated with bisulfite. The indicated region of the ICSBP-gene was amplified and cloned. Several independent inserts were sequenced and results are presented schematically. Circles mark the position of CpG-dinucleotides (empty: unmethylated; filled: methylated).

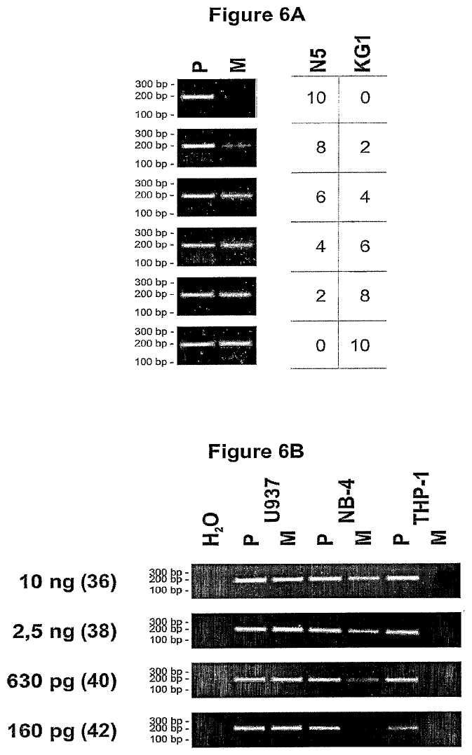

FIG. 6: Sensitivity of MB-PCR. (A) MB-PCRs for ESR1, CDKN2B (p15^{INK4b}), and ICSBP promoters from mixtures of DNA from a healthy donor (unmethylated) DNA and DNA from the cell line KG-1 (methylated in all three loci). (B) DNA from three cell lines was subjected to MB-PCR using the indicated amounts of DNA for the M-reaction (or half of the indicated amount for the P-reaction). With decreasing amounts of DNA, the number of amplification cycles during PCR (given in parenthesis) was increased. Also shown is a sample that did not include DNA (H₂O).

FIG. 7: FIG. 7 shows the nucleotide sequence of plasmid pMTBip/MBD2-Fc and the protein sequence (in bold) of the MBD2-Fc bifunctional protein which is encoded by plasmid pMTBip/MBD2-Fc.

The amino acid sequence of the MBD2-Fc bifunctional protein has the following features.
AA 1-28 (nt 851-934): Drosophila BiP secretion signal (leader peptide from pMT/BipN5-His vector):
AA 29-115 (nt 935-1196): AA 144-230 of human MBD2
AA 116-129 (nt 1196-1237): Flexible Linker (AAADPIEGRGGGGG)
AA 130-361 (1238-1933): AA99-330 of human IGHG1

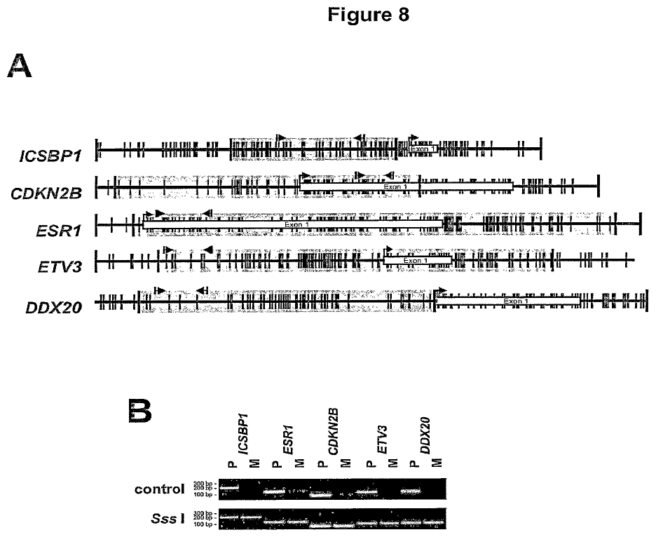

FIG. 8: MB-PCR detects methylation of CpG-island promoters (A) Schematic presentation of the detected MseI-fragments (indicated as grey boxes) of ESR1, CDKN2B (p15INK4b), ICSBP, ETV3, and DDX20. The position of CpG-dinucleotides, MseI-restriction sites, transcription start site, first exon and relative position of primers are marked. (B) Shown are representative MB-PCR results of normal (unmethylated) and in vitro methylated genomic DNA for the indicated promoters. The P-reaction directly amplifies the genomic DNA, whereas the M-reaction only amplifies CpGmethylated DNA fragments.

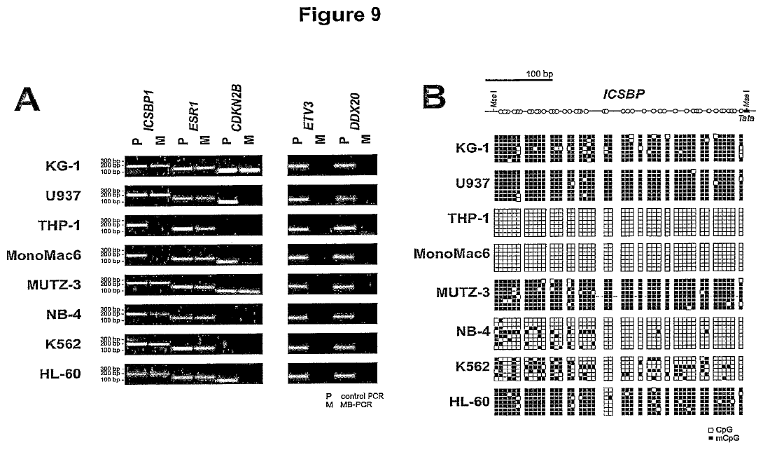

FIG. 9: Detecting CpG methylation in leukaemia cell lines by MB-PCR. (A) Shown are representative MB-PCR results of eight different leukaemia cell lines for the indicated promoters. (B) Genomic DNA from the same cell lines was analyzed by bisulfite sequencing. The indicated region of the ICSBP gene was amplified and cloned. Several independent inserts were sequenced and results are presented schematically. Squares mark the position of CpG-dinucleotides (empty: unmethylated; filled: methylated).

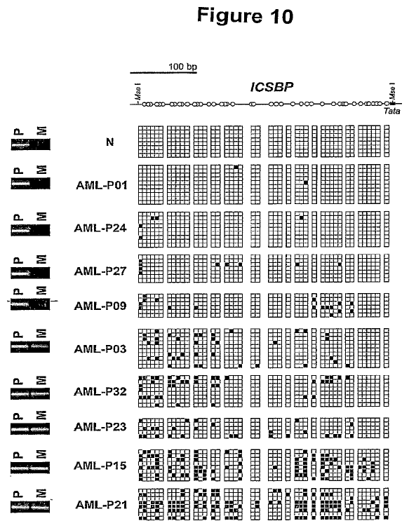

FIG. 10: Detection of aberrant CpG methylation in primary AML blasts. Two for the ICSBP promoter of one representative healthy donor (N) and nine AML patients are shown together with corresponding sequencing results. (Results of bisulfite sequencing are presented as described in FIG. 9.)

FIG. 11: Expression of MBD2-F_{c} in Drosophila Schneider-cells. Stably transfected S2 cells were seeded in Medium w/o FCS, with and w/o 500 μM CuSO₄. The supernatant was collected after 4 days and precleared o/n at 4° C. using sepharose beads. 1 ml precleared supernatant was precipitated using protein A sepharose, washed, re-suspended in SDS-loading dye and subjected to SDS-PAGE. The gel was Coomassie-stained to detect precipitated protein.

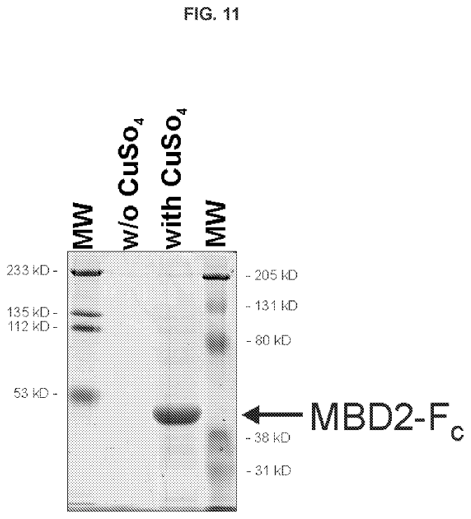

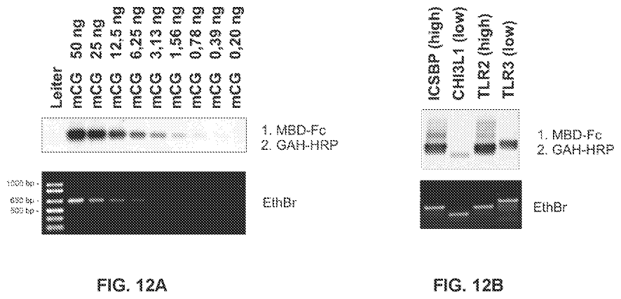

FIGS. 12A and 12B: Reverse South-Western Blot. A 650 bp PCR-fragment of human ICSBP-promoter (FIG. 12A) or methylated promoter fragments (50 ng) of varying CpG-density (FIG. 12B) (number of CpG-dinucleotides/100 bp: ICSBP: 10,6; CHI3L1: 2,9; TLR2: 6,2; TLR3: 2,1) were methylated using SssI, subjected to agarose gel electrophoresis (ethidium bromide staining is shown as control) and directly blotted onto nylon membrane. Membranes were stained using MBD2-Fc, HRP-conjugated anti-human Fc and ECL as described in Example 3.

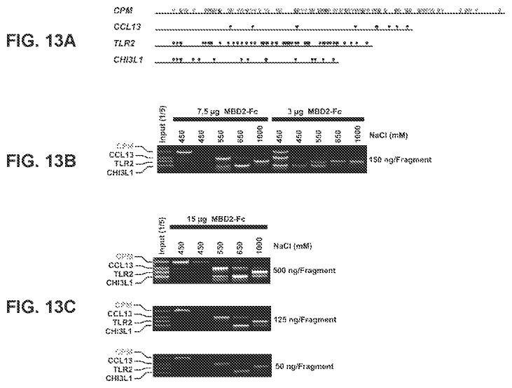

FIGS. 13A, 13B, and 13C. Salt concentration-dependent binding of CpG-methylated to MBD-Fc beads (FIG. 13A) Schematic presentation of human promoter fragments. Circles mark the position of CpG-dinucleotides (∘: unmethylated—CPM; ● SssI methylated—CCL13, TLR2, CHI3L1). (FIG. 13B and FIG. 13C) A mixture of methylated and un-methylated fragments were bound to MBD2-Fc-sepharose (amount of MBD2-Fc/50 protein A-sepharose is given) eluted using increasing salt concentrations, purified and separated using agarose gel electrophoresis (along with ⅕ of the Input mixture). Bands were visualized with ethidium bromide and scanned using a Typhoon Imager (Pharmacia-Amersham).

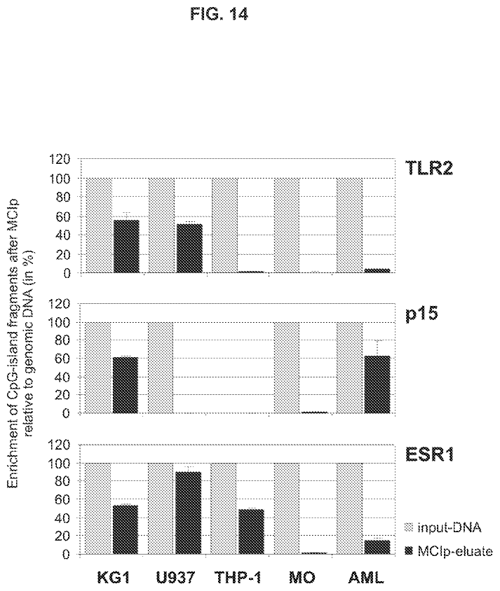

FIG. 14: Enrichment of CpG-islands by MCIp. Genomic DNA (300 ng) of the indicated cell types was subjected to MCIp. The enrichment of three CpG island promoters (TLR2, p15 and ESR1) was quantified using LightCycler real-time PCR. The amount of a particular promoter fragment amplified from the MCIp-eluate is shown relative to the untreated genomic DNA-control. The p15 promoter was undetectable in THP-1 cells indicating a mutation or deletion of this gene.

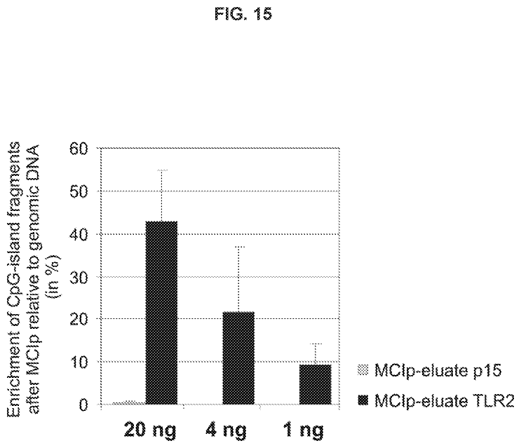

FIG. 15 Sensitivity of methylated CpG-island detection by MCIp. Decreasing amounts of restricted genomic U937 DNA was subjected to MCIp. The enrichment of the two CpG island promoters (TLR2, p15) was quantified using LightCycler real-time PCR. The amount of a particular promoter fragment amplified from the MCIp-eluate is shown relative to the untreated genomic DNA-control.

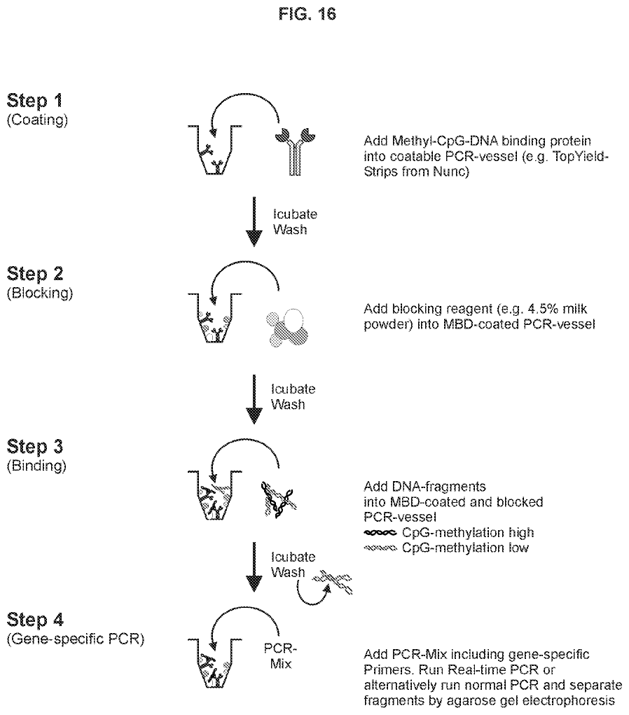

FIG. 16: Principle of MB-PCR. This figure shows a schematic representation of MB-PCR.

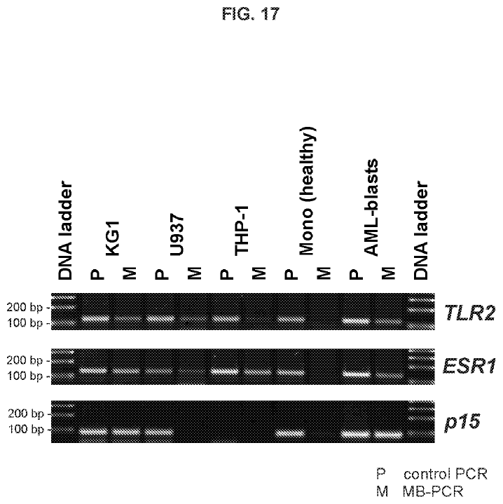

FIG. 17: MB-PCR of TLR2, ESR1 and p15 promoters in a normal and four leukemic DNA samples. Genomic DNA (10 ng) of the indicated cell types was subjected to MB-PCR. The enrichment of three CpG island promoters (TLR2, p15 and ESR1) was detected by standard genomic PCR. The p15 promoter was undetectable in THP-1 cells indicating a mutation or deletion of this gene.

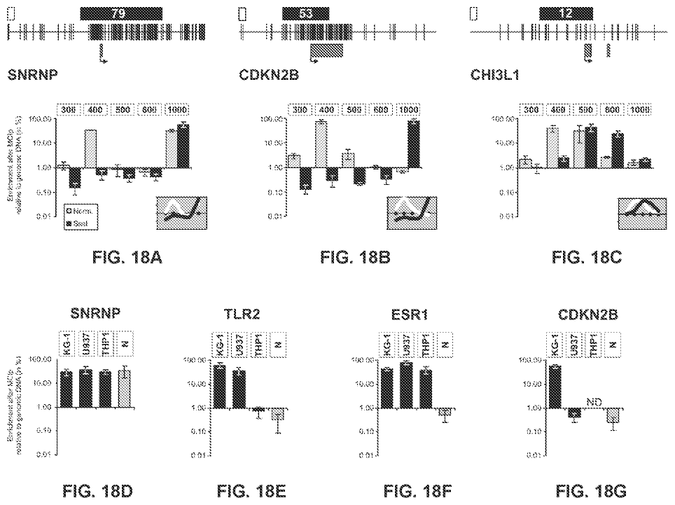

FIG. 18A-18G: MCIp detection of CpG methylation in specific CpG island promoters using real-time PCR. (FIG. 18A-C) Fractionated Methyl-CpG immunoprecipitation (MCIp) was used in combination with real-time LightCycler PCR to detect the methylation status of the indicated genes from untreated (gray bars) and SssI-methylated and MseI-restricted genomic DNA fragments (black bars). Recovered gene fragments from MCIp-eluates (NaCl-concentrations (in mM) are given in boxes above) and an equivalent amount of input-DNA were amplified by LightCycler-PCR. Values (mean±SD, n=4) of individual fractions represent the percentage of recovery and are calculated relative to the amount of PCR-product generated from the respective input-DNA (100%). Above each figure a 3 kB region of the corresponding CpG island is schematically presented. Each CpG dinucleotide is represented by a vertical line. The positions of exons are indicated as grey boxes and transcription start sites by an arrow. The white box represents a 100 bp fragment. Black boxes indicate the positions of the MseI-fragments that are detected. (FIG. 18D-G) SNRPN, TLR2, ESR1, and CDKN2B gene fragments in the high salt (1000 mM) MCIp fraction of three human myeloid leukaemia cell lines (KG-1, U937 and THP-1), as well as normal human blood monocytes (N) were analyzed by Real time PCR as above.

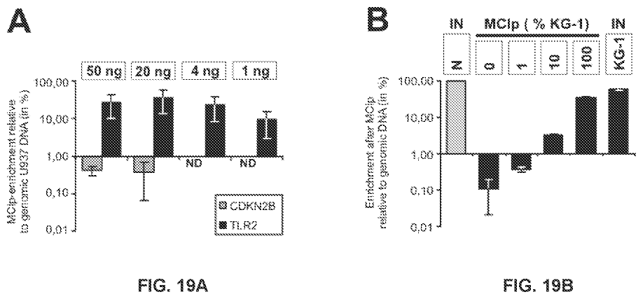

FIGS. 19A and 19B: Sensitivity and linearity of the MCIp approach. (FIG. 19A) Decreasing amounts of MseI-treated U937 DNA were subjected to MCIp. CDKN2B and TLR2 gene fragments were quantified as above. (FIG. 19B). MseI-treated DNA of normal human blood monocytes (N) and KG-1 cells was mixed at the indicated ratios and the mixture was subjected to MCIp and the TLR2 gene fragment was quantified using LightCycler-PCR as above

A better understanding of the present invention and of its many advantages will be seen from the following examples, offered for illustrative purposes only, and are not intended to limit the scope of the present invention in any way.

### EXAMPLE 1: SINGLE-TUBE ASSAY FOR THE DETECTION OF CPG-METHYLATED DNA-FRAGMENTS USING METHYL-BINDING POLYMERASE CHAIN REACTION (MB-PCR)

This method uses an approach similar to ELISAs. A protein with high affinity for CpG-methylated DNA is coated onto the walls of a PCR-cycler compatible reaction vessel and used to selectively capture strongly methylated DNA-fragments from a genomic DNA mixture. The retention of a specific DNA-fragment (e.g. a CpG island promoter of a specific gene) can be detected in the same tube using PCR (either standard PCR or realtime PCR, single or multiplex). The degree of methylation may be estimated relative to a PCR reaction of the genomic input DNA. FIG. 1 shows a schematic representation of MB-PCR.

### 1. Cells, Patient Samples, DNA Preparation and Fragmentation

### Cells

Peripheral blood mononuclear cells (MNC) were separated by leukapheresis of healthy donors, followed by density gradient centrifugation over Ficoll/Hypaque. Monocytes were isolated from MNC by countercurrent centrifugal elutriation in a J6ME centrifuge (Beckman, München, Germany) as described in Krause, J. Leukoc. Biol. 60 (1996), 540-545. Drosophila S2 cells were obtained from ATTC and cultured in Insect-Xpress medium (Bio Whittaker) containing 10% fetal calf serum (FCS; PAA) in an incubator at 21° C. The human myeloid leukaemia cell lines THP-1, NB-4, KG-1, K562, HL-60, and U937 were grown in RPMI 1640 medium supplemented with 10% FCS. The human myeloid leukaemia cell line Mono Mac 6 was grown RPMI 1640 medium plus 10% FCS and 1% OPI media supplement (Sigma). The human myeloid leukaemia cell line MUTZ-3 was maintained in αMEM plus 20% FCS and 10 ng/ml stem cell factor. For DNA-demethylation, U937 cells were treated with the indicated amounts of Decitabine (2-deoxy-5′-azacytidine, Sigma) for several days.

### Patient Samples

Fresh peripheral blood samples and bone marrow specimens from 35 patients with newly diagnosed and untreated de novo or secondary AML were used for the study. All patients were treated according to the protocol AMLCG-2000 of the German AML Cooperative Group. The study was approved by the Institutional Ethics Committee, and written informed consent was obtained from each patient before entering the study.

### DNA Preparation and Fragmentation

Genomic DNA from various cellular sources, including the cell lines described herein (e.g. KG1, U937, and THP-1), normal human monocytes (healthy donor) and frozen blast cells from a patient with AML were prepared using Blood and Cell Culture Midi Kit (Qiagen). Quality of the genomic DNA-preparation was controlled by agarose gel electrophoresis and DNA concentration was determined by UV spectrophotometry. Genomic DNA was digested with Mse I (NEB) and finally quantified using PicoGreen dsDNA Quantitation Reagent (Molecular Probes). Where indicated, DNA was in vitro methylated using Sss I methylase (NEB).

### 2. Generation of a Recombinant Methyl-CpG-Binding Polypeptide

A cDNA corresponding to the methyl-CpG binding domain (MBD) of human MBD2 (Genbank acc. no. NM 003927; AA 144-230) was PCR-amplified from reverse transcribed human primary macrophage total RNA using primers MBD2-Nhe_S (5′-AGA TGC TAG CAC GGA GAG CGG GAA GAG G-3′) (SEQ ID NO: 4) and MBD2-Not_AS (5′-ATC ACG CGG CCG CCA GAG GAT CGT TTC GCA GTC TC-3′) (SEQ ID NO: 5) and Herculase DNA Polymerase (Stratagene). Cycling parameters were: 95° C., 3 min denaturation; 95° C., 20 s, 65° C., 20 s, 72° C., 80 s amplification for 34 cycles; 72° C., 5 min final extension. The PCR-product was precipitated, digested with Not I/Nhe I, cloned into NotI/NheI-sites of Signal pIg plus vector (Ingenius, R&D Systems) and sequence verified resulting in pIg/MBD2-Fc (eukaryotic expression vector). To clone pMTBip/MBD2-Fc for recombinant expression in Drosophila S2 cells, the Apa I/Nhe I—fragment of pIg/MBD2-Fc containing the MBD of human MBD2 fused to the Fc-tail of human IgG1 was subcloned into Apa I/Spe I—sites of pMTBiP/V5-His B (Invitrogen).

Drosophila S2 cells were obtained from ATTC and cultured in Insect-Xpress medium (Bio Whittaker) containing 10% FCS (PAA) in an incubator at 21° C.

4×10⁶ Drosophila S2 cells/60 mm cell culture dish were transfected with a mixture of 1.5 μg pMTBip/MBD2-Fc and 0.3 μg pCoHygro (Invitrogen) using Effectene transfection reagent (Qiagen) according to the manufacturers protocol. On day three, transfected cells were harvested, washed, and replated in selection medium (Insect-Xpress) containing 10% FCS and 300 μg/ml Hygromycin (BD Biosciences). Selection medium was replaced every 4-5 days for five weeks. The pool of stably transfected Drosophila S2 cells was expanded. For large scale production of the methyl-CpG binding polypeptide MBD-Fc, 1-5×10⁸ cells were cultured in 100-200 ml Insect-Xpress without FCS (optional: 300 μg/ml Hygromycin) in 2000 ml roller bottles for two days before the addition of 0.5 mM CuSO₄. Medium was harvested every 4-7 days and cells were replated medium plus CuSO₄ for further protein production. Cell culture supernatants were combined, dialysed against TBS (pH 7.4), and purified using a protein A column. The MBD-Fc containing fractions were combined and dialysed against TBS (pH 7.4). The stably transfected Drosophila S2 cells produced 3-5 mg recombinant MBD2-Fc protein per litre cell culture supernatant. The sequence and features of the MBD-Fc protein are shown in FIG. 7.

### 3. Preparation of MB-PCR Tubes

50 μl of the recombinant MBD2-Fc protein comprising the methyl-CpG binding domain (MBD) of human methyl-CpG-binding domain 2 (MBD2), a flexible linker polypeptide and the Fc portion of human IgG1 (diluted at 15 μg/ml in 10 mM Tris/HCl pH 7.5) were added to each well of heat stable TopYield™ Strips (Nunc Cat. No. 248909) and incubated overnight at 4° C. Wells were washed three times with 200 μl TBS (20 mM Tris, pH 7.4 containing 170 mM NaCl) and blocked at RT for 3-4 h with 100 μl Blocking Solution (10 mM Tris, pH 7.5 containing 170 mM NaCl, 5% skim milk powder, 5 mM EDTA and 1 μg/ml of each poly d(I/C), poly d(A/T) and poly d(CG), all from Amersham). Tubes were washed three times with 200 μl TBST (TBS containing 0.05% Tween-20).

### 4. Binding of Methylated DNA Fragments

50 μl Binding Buffer (20 mM Tris, pH 7.5 containing 400 mM NaCl, 2 mM MgCl₂, 0.5 mM EDTA, and 0.05% Tween-20) were added to each well and 2 μl Mse I-digested DNA (5 ng/μl) was added to every second well (M-reaction). Wells were incubated on a shaker at RT for 40-50 min. Tubes were washed two times with 200 μl Binding Buffer and once with 10 mM Tris/HCl pH 8.0.

### 5. Detection of Methylated DNA Fragments

PCR was carried out directly in the treated and washed TopYield™ Strips. The PCR-mix (PCR Master Mix (Promega); 50 μl-reactions/well) included 10 pmol of each gene-specific primer (synthesized by Metabion). Primer sequences were P15 S (5′-GGC TCA GCT TCA TTA CCC TCC-3′) (SEQ ID NO: 6), P15 AS (5′-AAA GCC CGG AGC TAA CGA C-3′) (SEQ ID NO: 7), ESR1 S (5′-GAC TGC ACT TGC TCC CGT C-3′) (SEQ ID NO: 8), ESR1 AS (5′-AAG AGC ACA GCC CGA GGT TAG-3′) (SEQ ID NO: 9), ICSBP S (5′-CGG AAT TCC TGG GAA AGC C-3′) (SEQ ID NO: 10), ICSBP AS (5′-TTC CGA GAA ATC ACT TTC CCG-3′) (SEQ ID NO: 11), METS S (5′-AAT TGC GTC TGA AGT CTG CGG-3′), (SEQ ID NO. 12), METS AS (5′-TCC CAC ACA ACA GAG AGG CG-3′) (SEQ ID NO. 13), DP103 S (5′-GCT GTT AGT CCA GTT CCA GGT TCC-3′) (SEQ ID NO. 14), DP103 AS (5′-GTG CAA CCA CAT TTA TCT CCG G-3′) (SEQ ID NO: 15).

After adding the PCR-mix, 1 μl Mse I-digested DNA (5 ng/μl) was added to every other second well that was not previously incubated with DNA-fragments (P-reaction). PCR was performed on a MJResearch engine with the following cycling conditions: 95° C. for 3 min (denaturation), 94° C. for 20 s, 60° C. for 20 s, and 72° C. for 70 s (36 cycles) and 72° C. for 5 min (final extension). PCR-products were analyzed using 3% agarose gel electrophoresis and the ethidium bromide stained gel was scanned using a Typhoon 9200 Imager (Amersham/Pharmacia).

### 6. Sodium Bisulfite Sequencing

Modification of DNA with sodium bisulfite was performed as previously described. Bisulfite-treated DNA was amplified in a nested PCR reaction using the primers icsbp-out S (5′-GGG GTA GTT AGT TTT TGG TTG-3′) (SEQ ID NO: 16) and icsbp-out AS (5′-ATA AAT AAT TCC ACC CCC AC-3′) (SEQ ID NO: 17) for the first and icsbp-in S (5′-TTG TGG ATT TTG ATT AAT GGG-3′) (SEQ ID NO: 18) and icsbp-in AS (5′-CCR CCC ACT ATA CCT ACC TAC C-3′) (SEQ ID NO: 19) for the second round of amplification. PCR-products were cloned using TOPO-TA Cloning Kit (Invitrogen) and several independent clones were sequenced.

### 7. RNA-Preparation, Real-Time-PCR

Total RNA was isolated from different cell lines by the guanidine thiocyanate/acid phenol method (Chomczynski, Anal. Biochem. 162 (1987), 156-159. RNA (2 μg) was reverse transcribed using Superscript II MMLV-RT (Invitrogen). Real-time PCR was performed on a Lightcycler (Roche) using the Quantitect kit (Qiagen) according to the manufacturer's instructions. Primers used were: human ICSBP: sense 5′-CGT GGT GTG CAA AGG CAG-3′ (SEQ ID NO: 20), antisense 5′-CTG TTA TAG AAC TGC TGC AGC TCT C-3′ (SEQ ID NO: 21); human ACTB (β-Actin): sense 5′-TGA CGG GGT TCA CCC ACA CTG TGC CCA TCT A-3′ (SEQ ID NO: 22), antisense 5′-CTA GAA GCA TTT GTG GTG GAC GAT GGA GGG-3′ (SEQ ID NO: 23). Cycling parameters were: denaturation 95° C., 15 min, amplification 95° C., 15 s, 57° C., 20 s, 72° C., 25 s for 50 cycles. The product size was initially controlled by agarose gel electrophoresis and melting curves were analyzed to control for specificity of the PCR reactions. ICSBP data were normalized for expression of the housekeeping gene β-actin (ACTB). The relative units were calculated from a standard curve plotting 3 different concentrations of log dilutions against the PCR cycle number (CP) at which the measured fluorescence intensity reaches a fixed value. The amplification efficiency E was calculated from the slope of the standard curve by the formula: E=10^{-1/slope}. E_{ICSBP} was in the range of 1.87 to 1.98, E_{ACTB} ranged from 1.76 to 1.84. For each sample, data of 3 independent analyzes were averaged.

8. Analyzing the CpG Island Methylation Status of ESR1, CDKN2B (p15^{INK4b}) and ICSBP Promoters by MB-PCR

Several leukaemia cell lines were analyzed for their CpG island methylation status of ESR1, CDKN2B (p15^{INK4b}), and ICSBP promoters by MB-PCR. Genomic DNA was digested with Mse I. This enzyme was chosen because it is methylation-insensitive and cuts DNA into small fragments but leaves CpG islands relatively intact. Location of the gene-specific Mse I-fragments relative to the first intron of their respective genes as well as positions of gene-specific primers used for PCR are shown in FIG. 2A. All fragments were chosen to include the putative proximal promoter regions. A total of 10 ng of restricted DNA were used for the M-reaction and 5 ng of the same digested genomic DNA were used for the P-reaction. The result of a representative MB-PCR experiment from eight different leukaemia cell lines is shown in FIG. 2B. The ESR1 promoter was amplified to varying degrees in the M-reaction of all eight samples, which is in line with previous reports demonstrating its aberrant methylation in 86% of human haematopoietic tumors. The P-reaction for the CDKN2B (p15^{INK4b}) promoter failed completely in three cell lines (THP-1, NB-4, K562) suggesting mutations) or deletions on both alleles, which has also been demonstrated before. Two cell lines (KG-1 and MUTZ3) showed a positive M-reaction for the CDKN2B (p15^{INK4b}) promoter, whereas three cell lines (U937, MonoMac6, HL-60) were negative. The observed results were in good concordance with previously published methylation analyzes of ESR1 and CDKN2B (p15^{INK4b}) promoters in some of these cell lines. In some cases, P-reactions were weaker in comparison with other cell types, suggesting the loss or mutation of one allele (e.g. ESR1 in U937 cells). The ICSBP promoter was also amplified in M-reactions of six cell lines.

The degree and effect of ICSBP promoter methylation was analyzed to further validate the experimental potential of MB-PCR. Expression levels of ICSBP were analyzed in the eight leukaemia cell lines using LightCycler Real time PCR. As shown in FIG. 3A, mRNA expression levels inversely correlated with methylation degree as determined by MB-PCR. Treatment of U937 cells, which show a high degree of ICSBP promoter methylation with the demethylating agent Decitabine (5-Aza-2′Deoxycytidine) led to a marked dose- and time-dependent induction of ICSBP mRNA expression (s. FIG. 3B) indicating that the methylation-induced repression of ICSBP transcription is reversible in these cells.

To test whether MB-PCR is also able to detect the methylation of CpG island promoters in primary tumor cells, DNA was prepared from blood monocytes of healthy individuals (n=4) and blast cells of patients with AML (n=11), digested with Mse I, and subjected to MB-PCR. As shown in FIG. 4, no significant level of methylation was detected in the DNA of healthy donors, whereas most patients showed significant methylation in at least one of the three promoters analyzed.

To determine how MB-PCR results correlate with the exact pattern of CpG methylation at the ICSBP promoter, ICSBP promoter methylation was analyzed by bisulfate sequencing in selected cell lines, normal and tumor cells. The results shown in FIG. 5 indicate that the degree of promoter methylation can be predicted by MB-PCR—strong amplification signals appear to indicate a high degree, whereas weaker signals indicate a lesser degree of methylation.

Since patient samples may be contaminated with normal, potentially unmethylated cells, the effect of increasing amounts of normal DNA in a DNA sample of a tumor cell line was determined. Restricted DNA was mixed and subjected to MB-PCR. The results are shown in FIG. 6A. The signal in the M-reaction decreased in a linear fashion with increasing amounts of normal, unmethylated DNA in the sample. To test the sensitivity of the method, MB-PCR experiments using decreasing amounts of DNA were performed. As shown in FIG. 6B, comparable results were obtained using all concentrations tested (10 ng-160 pg) when analyzing the methylation status of the ICSBP locus in three different cell lines. These results indicate, that MB-PCR can detect methylated DNA-fragments in mixtures of normal cells, tumor cells, and works within the normal sensitivity range of standard genomic PCR (down to 160 pg of DNA).

9. Analyzing the CpG Island Methylation Status of ESR1, CDKN2B (p15^{INK4b}), ICSBP, ETV3, and DDX20 Promoters by MB-PCR,

In another experiment, the MB-PCR method was explored by analyzing the degree of CpG methylation of single CpG island promoters that were previously shown to be frequently methylated in leukaemia cells, namely the human CDKN2B gene (also known as p15INK4b) and the human estrogen receptor 1 (ESR1) gene. In addition to the well established tumor markers three additional genes with CpG island promoters that could potentially act as tumor suppressor genes were selected: the human interferon consensus binding protein (ICSBP) gene, the human Ets variant 3 gene (ETV3), and the human DEAD box polypeptide 20 gene (DDX20). ICSBP, a transcription factor of the interferon (IFN) regulatory factor family (IRF), is frequently down-regulated in human myeloid leukaemia (Schmidt, Blood 91 (1991), 22-29) and ICSBP-deficient mice display hematological alterations similar to chronic myelogenous leukaemia (CML) in humans (Holtschke, Cell 87 (1996), 307-317), suggesting a tumor suppressor function for ICSBP in hemopoietic cells. In mice, the Ets repressor ETV3 (also known as METS or PE1) and its co-repressor DDX20 (also known as DP103) were shown to link terminal monocytic differentiation to cell cycle arrest (Klappacher, Cell 109 (2002), 169-180), which may also indicate a possible tumor suppressor role. As a validation of our approach, genomic DNA from normal cells was either left untreated or methylated in vitro using SssI, digested with MseI and subjected to MB-PCR. Genomic DNA was digested with MseI because this enzyme is methylation-insensitive and cuts DNA into small fragments while leaving CpG islands relatively intact (Cross, Nat. Genet. 6 (1994), 236-244). Locations of the gene-specific MseI-fragments relative to the first intron of their respective genes as well as positions of gene-specific primers used for MB-PCR are shown in FIG. 8A. All fragments include the putative proximal promoter regions. As shown in FIG. 8B, the M-reactions of all five loci were negative when normal DNA was used, indicating that these genomic regions are, as expected, free of methylation in normal blood cells. However, each locus was amplified in the corresponding M-reaction when the same DNA was in vitro methylated using SssI-methylase before it was subjected to MB-PCR. Hence, MB-PCR is able to discriminate the methylated and unmethylated state at these loci.

### 10. Methylation Status of Specific CpG Island Promoters in Tumor Cell Lines Analyzed by MB-PCR.

In another experiment it was tested whether MB-PCR is able to detect the methylation status of the above loci in biological samples, several leukaemia cell lines were analyzed. Routinely, a total of 10 ng of restricted DNA was used for the M-reaction and 5 ng of the same digested genomic DNA was used for the P-reaction. The result of a representative MB-PCR experiment from eight different leukaemia cell lines is shown in FIG. 9A. The ESR1 promoter was amplified to varying degrees in the M-reaction of all eight samples, which is in line with previous reports demonstrating its aberrant methylation in more than 80% of human hemopoietic tumors. The P-reaction for the CDKN2B promoter failed completely in three cell lines (THP-1, NB-4, K562) suggesting mutations or deletions on both alleles, which has been demonstrated previously in the cases of NB-4 (Chim, Ann. Hematol. 82 (2003), 738-742) and K562 (Paz, Cancer Res. 63 (2003), 1114-1121). The two cell lines KG-1 and MUTZ3 showed a positive M-reaction for the CDKN2B promoter, whereas three cell lines (U937, MonoMac6, HL-60) were negative. The observed results were in good concordance with previously published methylation analyzes of ESR1 (27) and CDKN2B promoters (Cameroon, Blood 94 (1999), 2445-2451; Chim (2003), loc. cit.; Paz (2003), loc. cit.). In some cases, P-reactions were weaker in comparison with other cell types, suggesting the loss or mutation of one allele (e.g. ESR1 in U937 cells).

Interestingly, the ICSBP promoter was also amplified in M-reactions of six cell lines, whereas no significant methylation was detected at the promoters of ETV3 and DDX20 genes.

To determine how MB-PCR results correlate with the exact pattern of CpG methylation at the ICSBP promoter in individual cell lines, the ICSBP promoter methylation was analyzed by bisulfite sequencing. The results shown in FIG. 9B indicate that the degree of promoter methylation corresponds with results obtained by MB-PCR. Strong amplification signals (comparable to the corresponding P-reaction), as seen in KG-1, U937, MUTZ-3, HL-60, and K562 cell lines, appear to indicate a high degree, whereas weaker signals (as observed for NB-4 cells) indicate a lesser degree of methylation. In the absence of DNA methylation (THP-1 and MonoMac6 cells) the MB-PCR is negative.

### 11. Detecting Methylation of CpG Island Promoters in Primary Tumor Cells.

DNA was prepared from blood monocytes of several healthy persons (n=4) and leukaemic blasts of patients with previously untreated AML (n=35), digested with MseI, and subjected to MB-PCR. FIG. 11 shows representative ICSBP MB-PCR and corresponding bisulfite sequencing results for 9 AML patients and 1 normal individual. In general, the intensity of the band observed in the M-reaction (as compared to the corresponding P-reaction) showed good correlation with the mean density of methylation in the sample. Out of 35 AML-patients tested, 7 patients (20%) showed positive MB-PCR results for ICSBP, 21 patients (60%) for ESR1 and 25 patients (71%) for CDKN2B (data not shown). The frequencies for ESR1 and CDKN2B methylation observed concur with those described in previous studies. ICSBP methylation apparently only affects a subgroup of patients. Twelve patients were tested for methylation of ETV3 and DDX20 genes and, as observed for the leukaemia cell lines, no significant methylation was detected in any of the samples.

### EXAMPLE 2: CLONING OF PMTBIP/MBD2-FC

A cDNA corresponding to the methyl-CpG binding domain (MBD) of human MBD2 (Genbank acc. no. NM 003927; AA 144-230) was PCR-amplified from reverse transcribed human primary macrophage total RNA using primers MBD2-Nhe_S (5′-AGA TGC TAG CAC GGA GAG CGG GAA GAG G-3′) (SEQ ID NO: 4) and MBD2-Not_AS (5′-ATC ACG CGG CCG CCA GAG GAT CGT TTC GCA GTC TC-3′) (SEQ ID NO: 5) and Herculase DNA Polymerase (Stratagene). Cycling parameters were: 95° C., 3 min denaturation; 95° C., 20 s, 65° C., 20 s, 72° C., 80 s amplification for 34 cycles; 72° C., 5 min final extension. The PCR-product was precipitated, digested with Not I/Nhe I, cloned into NotI/NheI-sites of Signal pIg plus vector (Ingenius, R&D Systems), and sequence verified resulting in pIg/MBD2-Fc (eucaryotic expression vector). To clone pMTBip/MBD2-Fc for recombinant expression in Drosophila S2 cells, the Apa I/Nhe I—fragment of pIg/MBD2-Fc containing the MBD of human MBD2 fused to the Fc-tail of human IgG1 was subcloned into Apa I/Spe I—sites of pMTBiP/V5-His B (Invitrogen).

### EXAMPLE 3: RECOMBINANT EXPRESSION OF AN ANTIBODY-LIKE METHYL-CPG-DNA-BINDING PROTEIN

Methylated Cytosine in single-stranded, but not double-stranded DNA molecules can be efficiently detected using 5-mC antibodies. To enable an antibody-like detection of double-stranded CpG-methylated DNA, a vector as described in Example 2 above, was constructed encoding a fusion protein comprising the methyl-CpG binding domain (MBD) of human methyl-CpG-binding domain 2 (MBD2), a flexible linker polypeptide, and the Fc portion of human IgG1. The protein was expressed under the control of a metal-inducible promoter in Drosophila S2 Schneider-cells, and collected from the supernatant via Protein A affinity chromatography. The purified protein was expressed in high amounts (4-5 mg/L cell culture supernatant) and had the expected molecular weight of appr. 40 kDa (s. FIG. 2).

Accordingly, in detail an insect cell system was chosen for recombinant expression of MBD2-Fc protein for several reason. The main reason is the absence or low abundance of CpG-methylation. Production of the protein in mammalian (especially human) cells may result in DNA contaminations (bound to the MBD2-Fc protein in the cell culture supernatant) which may complicate subsequent analysis of CpG-methylated DNA. Other reasons include the simple culture conditions and the potentially high yields of protein.

Drosophila S2 cells were obtained from ATTC and cultured in Insect-Xpress medium (Bio Whittaker) containing 10% FCS (PAA) in an incubator at 25° C.

4×10⁶ Drosophila S2 cells/60 mm cell culture dish were transfected with a mixture of 1.5 μg pMTBip/MBD2-Fc and 0.3 μg pCoHygro (Invitrogen) using Effectene transfection reagent (Qiagen) according to the manufacturers protocol. On day three, transfected cells were harvested, washed, and replated in selection medium (Insect-Xpress) containing 10% FCS and 300 μg/ml Hygromycin (BD Biosciences). Selection medium was replaced every 4-5 days for five weeks. The pool of stably transfected Drosophila S2 cells was expanded and several aliquots preserved in liquid nitrogen.

For large scale production, 1-5×10⁸ cells were cultured in 100-200 ml Insect-Xpress without FCS (optional: 300 μg/ml Hygromycin) in 2000 ml roller bottles for two days before the addition of 0.5 mM CuSO₄. Medium was harvested every 4-7 days, and cells were replated medium plus CuSO₄ for further protein production. Cell culture supernatants were combined, dialysed against TBS (pH 7.4) and purified using a protein A column. The MBD-Fc containing fractions were combined and dialysed against TBS (pH 7.4). The stably transfected Drosophila S2 cells produced 3-5 mg recombinant MBD2-Fc protein per litre cell culture supernatant.

### EXAMPLE 4: DETECTION OF CPG-METHYLATED DNA ON MEMBRANES (REVERSE SOUTH-WESTERN BLOT)

To test, whether MBD2-Fc was able to detect CpG-methylated DNA on membrane in a Western blot-like procedure, we blotted in vitro methylated or unmethylated PCR-fragments with different CpG density onto a Nylon-membrane using a capillary transfer system equivalent to traditional Southern blotting, however without denaturing the DNA prior to blotting. As shown in FIG. 12, using standard immunoblot conditions and MBD-Fc as an equivalent to the primary antibody, methylated DNA can be detected on Nylon membranes in a linear fashion (FIG. 12A) and depending on the CpG content (FIG. 12B). These results indicated that the MBD-Fc fusion protein is able to detect CpG-methylated DNA bound to a solid support.

### EXAMPLE 5: SMALL SCALE ENRICHMENT OF CPG-METHYLATED DNA USING METHYL-CPG-IMMUNOPRECIPITATION (MCIP)

The following protocol allows a quick enrichment of CpG-methylated DNA fragments using spin columns. The DNA is bound to MBD2-Fc protein coupled to Sepharose beads via Protein A. The affinity for methylated DNA increases with the density of methylated CpG-dinucleotides and decreases with the ionic strength of the wash buffer.

### 5.1 Binding of the MBD2-Fc Protein to Protein a Sepharose

8-10 μg purified MBD2-Fc protein was added to 50 μl Protein A Sepharose 4 Fast Flow beads (Amersham) in 1 ml TBS and rotated over night on a rotator at 4° C. On the next day, MBD2-Fc-beads were washed twice with buffer A (20 mM Tris-HCl pH 8.0, 2 mM MgCl₂, 0.5 mM EDTA, 150 mM NaCl, 0.1% NP-40).

### 5.2 Restriction Digest and Quantitation of DNA

At least 1 μg genomic DNA (prepared using Qiagen columns) was digested using Mse I. Complete digest was controlled using agarose gel elecrophoresis and digested DNA was exactly quantified using PicoGreen dsDNA Quantitation Reagent (Molecular Probes).

### 5.3 Purification of Highly Methylated CpG-DNA

Digested DNA (300 ng) was added to the washed MBD2-Fc-beads in 1 ml buffer A and rotated for 3 h on a rotator at 4° C. Beads were transferred into SpinX-columns and spin-washed with approximately 1 ml buffer A. Beads were washed twice with 400 μl buffer B (20 mM Tris-HCl pH 8.0, 2 mM MgCl₂, 0.5 mM EDTA, 450 mM NaCl, 0.1% NP-40) and twice with buffer C (20 mM Tris-HCl pH 8.0, 2 mM MgCl₂, 0.5 mM EDTA, 650 mM NaCl, 0.1% NP-40). Flow through of each wash step was either discarded or collected for further analyzes. CpG-methylated DNA was eluted with 250 μl buffer D (20 mM Tris-HCl pH 8.0, 2 mM MgCl₂, 0.5 mM EDTA, 1000 mM NaCl, 0.1% NP-40) into a new tube. Eluted DNA was desalted using Qiaquick Spin columns (ELUTED). In parallel, 300 ng digested DNA (INPUT) was resuspended in 250 μl buffer D and desalted using the QIAquick PCR Purification Kit (Qiagen). Both ELUTED- and INPUT-DNA was exactly quantified using the PicoGreen dsDNA Quantitation Reagent (Molecular Probes).

### 5.4. Alternative Approaches

DNA may be restricted using different restriction endonucleases or by sonication.

### EXAMPLE 6: DETECTION AND QUANTITATION OF METHYLATED CPG-DNA FRAGMENTS GENERATED BY MCIP

To test, whether the MBD-Fc fusion protein was able to bind CpG-methylated DNA fragments in an immunoprecipitation-like approach, we first tested the binding properties of in vitro generated and differentially methylated DNA-fragments. PCR fragments of human promoters with varying CpG-density were generated using PCR (see FIG. 13) and CpG-methylated using SssI (CCL13, TLR2, CHI3L1) or left un-methylated (CPM). DNA was bound to MBD-Fc-Protein A sepharose beads in 150 mM NaCl (see. Example 5) and eluted using increasing concentrations of NaCl. Fractions were collected, spin-purified, and subjected to agarose gel electrophoresis. As shown in FIG. 13B, the affinity of a methylated fragment increased with the density of methylated CpG-dinucleotide with unmethylated DNA (CPM promoter fragment) eluting at relatively low salt concentrations and highly methylated DNA (TLR2 promoter fragment) eluting at high salt concentrations. Variation of the amount of Input-DNA did not significantly change the elution profile. However, the salt-dependent affinity of DNA was dependent on the density of the MBD-Fc fusion protein on the protein A sepharose beads. These results indicated that the MBD-Fc fusion protein is able to capture and bind CpG-methylated DNA in solution in a salt concentration- and CpG-methylation density-dependent fashion.

### 6.1 Quantitation on Single Gene Level Using Gene-Specific Real-Time PCR

6.1.1 To test whether the recombinant MBD-Fc protein was able to detect the methylation density of a CpG island promoter in a complex genomic DNA mixture, genomic DNA from three leukemia cell lines and normal donor monocytes as well as blast cells from a patient with AML were restricted with Mse I and subjected to MCIp. The enrichment of three CpG island promoters (TLR2, p15 and ESR1) in the 1000 mM NaCl MCIp-fraction was detected using LightCycler-PCR. The three loci were chosen because p15 and ESR1 are known targets for methylation in leukemia and TLR2 was previously shown to be methylated in U937 cells but not in THP-1 cells. As shown in FIG. 14, none of the three loci was significantly detectable in the DNA preparation from the normal donor DNA (MO), which is consistent with a usually unmethylated state of CpG island promoters in normal cells. The enrichment of TLR2 in U937 but not in THP-1 is consistent with the previously observed methylation pattern in both cells. Bisulfite sequencing of the TLR2 promoter as described in Hähnel, J. Immunol. 168 (2002), 5629-37) demonstrated an almost complete methylation of the TLR2 promoter in KG1-cells (data not shown) which is consistent with the strong MCIp-enrichment shown in FIG. 14. The results for p15 in KG1 and U937 are consistent with published data. These data indicate that MCIp can be used to detect methylated DNA fragments of single gene fragments in genomic DNA.

Accordingly, enrichment of a specific Mse I-fragment in the MCIp eluate was detected and quantified relative to the genomic INPUT by Real-time Lightcycler-PCR. (s. FIG. 14). The enrichment may also be quantified after an unspecific DNA-amplification of both ELUTED- and INPUT-DNA (s. amplicon generation in Example 6.2.1 below, data not shown).

TABLE 3
Gene-specific oligonucleotide primers for CpG-
island promoters

| | Mse I | | | |
|---|---|---|---|---|
| | fragment | | Antisense | product |
| Gene | (bp) | Sense primer | primer | (bp) |
| TLR2 | 1358 | TGTGTTTCAGGT | CGAATCGAGACGC | 118 |
| | | GATGTGAGGTC | TAGAGGC | |
| p15 | 699 | GGCTCAGCTTCA | AAAGCCCGGAGCT | 87 |
| | | TTACCCTCC | AACGAC | |
| ESR1 | 1108 | GACTGCACTTGC | AAGAGCACAGCCC | 129 |
| | | TCCCGTC | GAGGTTAG | |

In order to test whether MCIp may be used to discriminate methylated and unmethylated DNA fragments from genomic DNA, MCIp was used to enrich MseI-restricted genomic DNA of in vitro SssI-methylated and untreated normal DNA from monocytes of a healthy donor. MseI was chosen for DNA fragmentation, because it is known to preferentially cut in regions of low CpG content while leaving many CpG islands uncut (Cross, Nat. Genet. 6 (1994), 236-244).

The salt concentration-dependent enrichment of four different CpG-island promoters and a promoter with low CpG density was determined in SssI-methylated and untreated DNA relative to the input-DNA using LightCycler real-time PCR. As a positive control for DNA methylation, the SNRPN gene promoter that is subject to maternal imprinting with one of its two copies being methylated also in normal cells (Zeschnigk, Hum. Mol. Genet. 6 (1997), 387-395) was used. In normal DNA the two differentially methylated allele-fragments of SNRPN were enriched in two separate fractions (s. FIG. 18A). Only one enriched fraction was observed with SssI-methylated DNA. In the case of CDKN2B gene (also known as p15^{INK4b}) which is known to be frequently methylated in leukaemia cells (Chim, Ann. Hematol. 82 (2003), 738-742; Dodge, Int. J. Cancer 78 (1998), 561-567; Dodge, Leuk. Res. 25 (2001), 917-925) (FIG. 18B), the fragment was detected mainly in a low salt fraction from normal DNA and in the high salt fraction from SssI-methylated DNA. Similar results were obtained for the human estrogen receptor 1 (ESR1) gene (Issa, Cancer Res. 56 (1996), 973-977) and the human Toll-like receptor 2 gene (TLR2) (data not show). As shown in FIG. 18C, the profiles of methylated and unmethylated DNA at the CHI3L1 locus were significantly different from those of the above tested CpG island promoters. Most of the untreated CHI3L1-fragment was recovered at lower NaCl concentrations, and a slight shift was observed towards higher NaCl concentrations when the DNA was SssI-methylated. Analysis of the above elution profiles suggests that:
a.) A two to three hundred-fold enrichment of stronger over less methylated genomic fragments can be obtained in either low or high salt fractions;
b.) Fragments with low CpG density are largely excluded from the high salt fraction.
c.) The fractionated MCIp approach allows the resolution of small differences in CpG methylation density (the average difference between SssI-treated and untreated monocyte DNA is approximately six out of twelve methylated CpG residues, data not shown);

In order to test whether MCIp can detect aberrant hypermethylation in tumor samples, DNA from three leukaemia cell lines (KG1, U937, THP-1), as well as from monocytes of a healthy donor, were analyzed for SNRNP, CDKN2B, ESR1, and TLR2 promoter enrichment in the high salt fraction (s. FIG. 18D-G). The TLR2 gene promoter was enriched in KG-1 and U937 cells, but not in THP-1 or normal cells. The methylation pattern of TLR2 was confirmed by bisulfite sequencing (Haehnel, J. Immunol. 168 (2002), 5629-5637) (data not shown). Results for CDKN2B (KG-1 and U937) and ESR1 (KG-1) were also in line with previously published studies (Chim (2003); Dodge (2001); Issa (1996), all loc. cit.). None of the above three MseI fragments was significantly enriched in the DNA from normal cells. In concordance with its imprinting-related methylation status, the SNRPN gene promoter was significantly enriched in all leukaemia cell lines as well as in normal cells. These experiments established that the high salt MCIp fraction specifically enriches genomic DNA-fragments with a high degree of CpG methylation.

TABLE 4
Gene-specific oligonucleotide primers for
real-time amplification of CpG-island promoters

| Gene | Primer sequence (sense & antisense) |
|---|---|
| SNRNP | 5′-TAC ATC AGG GTG ATT GCA GTT CC-3′ |
| | 5′-TAC CGA TCA CTT CAC GTA CCT TCG-3′ |
| TLR2 | 5′-TGT GTT TCA GGT GAT GTG AGG TC-3′ |
| | 5′-CGA ATC GAG ACG CTA GAG GC-3 |
| ESR1 | 5′-GAC TGC ACT TGC TCC CGT C-3′ |
| | 5′-AAG AGC ACA GCC CGA GGT TAG-3′ |
| CDKN2B | 5′-GGC TCA GCT TCA TTA CCC TCC-3′ |
| | 5′-AAA GCC CGG AGC TAA CGA C-3′ |
| CHI3L1 | 5′-ATC ACC CTA GTG GCT CTT CTG C-3′ |
| | 5′-CTT TTA TGG GAA CTG AGC TAT GTG TC-3′ |

6.1.2. In order to determine the amount of DNA required for the detection of a single gene fragment in a complex mixture of genomic DNA, decreasing amounts of DNA fragments were subjected to MCIp and subsequent LightCycler real-time PCR. As shown in FIG. 15, the methylated TLR2 promoter can be enriched and detected from as little as 1 ng genomic DNA from U937 cells. The un-methylated p15-promoter was not significantly enriched (20 ng MCIp-eluate) or not detectable (4 ng or 1 ng MCIp-eluate) in U937 cells (FIG. 15). These results indicate that MCIp is a sensitive method to detect methylated DNA-fragments in a complex genomic mixture.

In order to test the sensitivity of the approach, decreasing amounts of U937 DNA were analyzed using the MCIp approach. The enrichment of TLR2 (strong methylation) and CDKN2B gene fragments (no methylation) were determined by LightCycler real-time PCR. As shown in FIG. 19A, a significant enrichment of the TLR2 fragment was achieved using as little as 1 ng of genomic DNA fragments (equivalent to approximately 150 tumor cells) for the MCIp procedure. Samples derived from tumors may contain significant numbers of normal cells that would be expected to be unmethylated at most CpG islands. To test how linear the detection of CpG methylation is with respect to cell purity, MCIp was performed using mixtures of DNA from normal blood cells and the leukaemia cell line KG-1 showing high levels of CpG island methylation at several promoters. As shown in FIG. 19B, the TLR2 promoter fragment was only detected in samples containing KG-1 DNA and the signal gradually increased with the proportion of methylated DNA in the sample. Similar results were obtained for the ESR1 locus (data not shown). In general, most informative (with respect to effects on transcription) and clearest results (in terms of noise and background) were obtained when a target gene fragment contained only the proximal promoter within the CpG island. Also, in addition to enzyme restriction, DNA fragmentation may also be achieved by mechanical means, e.g. sonication (data not shown).

### 6.2 Quantitation on Genome-Wide Level Using Microarray Technology

6.2.1 Generation of DNA-Amplicons from Genomic Mse I-Fragments Using Ligation-Mediated (Lm)-PCR

To generate a Mse I-compatible LMPCR-Linker, oligonucleotides LMPCR_S-L (5′-GCG GTG ACC CGG GAG ATC TCT TAA G-3′) and LMPCR_AS-L (5′-TAC TTA AGA GAT C-3′) were annealed as follows. Both oligos were combined at a concentration of 20 μM in nuclease-free H₂O (USB), incubated at 80° C. for 10 min, and cooled down slowly to RT. The annealed Linker was stored in 50 μl-aliquots at −20° C.

LMPCR-Linker (0.5 μl/ng ELUTED- or INPUT-DNA) was ligated to the ELUTED- and in a separate reaction to an equal amount of INPUT-DNA in 60 μl reactions using 1 μl T4-Ligase (1200 u/μl, NEB) at 16° C. o/n. Linker-ligated DNA was desalted using QIAquick PCR Purification Kit (Qiagen) and eluted in 55 μl Tris-HCl pH 8.0 (5 mM).

Linker-ligated DNA (ELUTED- and INPUT separately) was PCR-amplified using LMPCR-Primer (5′-GTG ACC CGG GAG ATC TCT TAA G-3′) and Taq DNA Polymerase (Roche). The PCR mix contained 25 μl 10×PCR-buffer (Roche), 15 μl MgCl₂ (25 mM, Roche), 10 μl dNTPs (10 mM each) 65 μl Betain (5M, Sigma), 2.5 μl LMPCR-Primer, 45 μl of linker-ligated DNA, 2.5 μl Taq DNA Polymerase (5 U/μl) in a total volume of 250 μl which was distributed into five PCR-tubes. Cycling parameters were: 58° C., 2 min (melting off LMPCR_AS-L), 72° C. 5 min (fill in overhangs); 95° C., 30 s, 58° C., 30 s, 72° C., 3 min amplification for 15 cycles; 72° C., 10 min final extension.

PCR-Reactions were combined and purified using QIAquick PCR Purification Kit (Qiagen). Both ELUTED- and INPUT-amplicons were exactly quantified using PicoGreen dsDNA Quantitation Reagent (Molecular Probes).

### 6.2.2. Analysis of MCIP-Amplicons Using CpG-Island Microarrays

MCIp-Amplicons may be analyzed using PCR (LightCycler, Standard PCR) to detect the enrichment of single gene fragments. To detect multiple gene fragments array technology may be used. The analysis of MCIp-amplicons using for example CpG island microarrays will involve the fluorescent labelling of MCIp-DNA-fragments and subsequent hybridization to microarrays using standard protocols.

### EXAMPLE 7: SINGLE-TUBE ASSAY FOR THE DETECTION OF CPG-METHYLATED DNA-FRAGMENTS USING METHYL-BINDING POLYMERASE CHAIN REACTION (MB-PCR)

This method uses an approach similar to ELISAs. A protein with high affinity for CpG-methylated DNA is coated onto the walls of a PCR-cycler compatible reaction vessel and used to selectively capture strongly methylated DNA-fragments from a genomic DNA mixture. The retention of a specific DNA-fragment (e.g. a CpG island promoter of a specific gene) can be detected in the same tube using PCR (either standard PCR or realtime PCR, single or multiplex). The degree of methylation may be estimated relative to a PCR reaction of the genomic input DNA. FIG. 16 shows a schematic representation of MB-PCR.

### 7.1 DNA Preparation and Fragmentation

Genomic DNA from three cell lines (KG1, U937, and THP-1), normal human monocytes (healthy donor) and frozen blast cells from a patient with AML were prepared using Blood and Cell Culture Midi Kit (Qiagen). Quality of the genomic DNA-preparation was controlled by agarose gel electrophoresis and DNA concentration was determined by UV spectrophotometry. Genomic DNA was digested with Mse I (NEB) and finally quantified using PicoGreen dsDNA Quantitation Reagent (Molecular Probes).

### 7.2 Preparation of PCR Tubes

MBD-Fc-coated PCR tubes were prepared using heat stable TopYield™ Strips (Nunc Cat. No. 248909). 50 μl of recombinant MBD-Fc protein (diluted at 15 μg/ml in 10 mM Tris/HCl pH 7.5) were added to each well and incubated overnight at 4° C. Wells were washed three times with 200 μl TBS (20 mM Tris, pH 7.4 containing 150 mM NaCl) and blocked overnight at 4° C. with 100 μl Blocking Solution (10 mM Tris, pH 7.5 containing 150 mM NaCl, 4.5% skim milk powder, 5 mM EDTA, and 0.8 μg/ml of each poly d(I/C), poly d(A/T and poly d(CG)). Tubes were washed three times with 200 μl TBST (TBS containing 0.1% Tween-20.

### 7.3 Binding of Methylated DNA

50 μl Binding Buffer (20 mM Tris, pH 7.5 containing 400 mM NaCl, 2 mM MgCl₂, 0.5 mM EDTA, and 0.1% Tween-20) were added to each well, and 1 μl Mse I-digested DNA (10 ng/μl) was added to every second well (M-reaction). Wells were incubated on a shaker at 4° C. for 3 hours. Tubes were washed three times with 200 μl Binding Buffer and once with 10 mM Tris/HCl pH 7.5.

### 7.4 Detection of Methylated DNA Fragments

PCR was carried out directly in the TopYield™ Strips. The PCR-Mix (50 μl/well) contained a standard PCR buffer (Roche), 2.5 U FastStart Taq DNA Polymerase (Roche), 10 pmol of each gene-specific primer (synthesized by Qiagen), dNTPs (200 mM each, Amersham/Pharmacia) 1 M betaine (Sigma), primer sequences, and cycling parameters are shown in Table 5 & 6, respectively. After adding the PCR-mix, 1 μl Mse I-digested DNA (10 ng/μl) was added to every second other well, that was not previously incubated with DNA-fragments (P-reaction). PCR-products were analyzed using agarose gel electrophoresis, and the ethidium bromide stained gel was scanned using a Typhoon 9200 Imager (Amersham/Pharmacia).

TABLE 5
Cycling parameters (MB-PCR):

| | | | |
|---|---|---|---|
| 94° C. | 3 | min | |
| 94° C. | 30 | s | |
| 60° C. | 30 | s | 37 × |
| 72° C. | 50 | s | |
| 72° C. | 5 | min | |

| | |
|---|---|
| 15° C. | ∞ |

TABLE 6
Gene-specific oligonucleotide primers
for CpG-island promoters

| | Mse I | | | |
|---|---|---|---|---|
| | fragment | | Antisense | product |
| Gene | (bp) | Sense primer | primer | (bp) |
| TLR2 | 1358 | TGTGTTTCAGGT | CGAATCGAGACGC | 118 |
| | | GATGTGAGGTC | TAGAGGC | |
| p15 | 699 | GGCTCAGCTTCA | AAAGCCCGGAGCT | 87 |
| | | TTACCCTCC | AACGAC | |
| ESR1 | 1108 | GACTGCACTTGC | AAGAGCACAGCCC | 129 |
| | | TCCCGTC | GAGGTTAG | |

FIG. 17 shows the result of an MB-PCR experiment analyzing the methylation profile of three different CpG-island promoters in five cell types. The lanes marked with P represent the amplification of the genomic input DNA. With an exception of the (probably deleted or mutated) p15 gene in THP-1 cells, all promoters were amplified. Notably, none of the promoters was detected in the MB-PCR reactions from the normal DNA control, which is consistent with the fact that these promoters are not methylated in normal individuals. In the cell lines as well as in the patient sample, promoters were mostly methylated. The results correspond to the data obtained with MCIp in independent experiments.

1. An in vitro method for detecting methylated DNA comprising:
(a) contacting a reagent capable of specifically binding methylated DNA with a sample comprising methylated and/or unmethylated DNA, wherein the reagent has been coated on a container; wherein the reagent comprises
(i) a first polypeptide and a second polypeptide each comprising a methyl-DNA-binding domain of an MBD2 protein, a fragment of the first polypeptide and a fragment of the second polypeptide, wherein each fragment is capable of binding methylated DNA, or a polypeptide that is at least 70% homologous to the first polypeptide or fragment thereof and is capable of binding methylated DNA and a polypeptide that is at least 70% homologous to the second polypeptide or the fragment thereof and is capable of binding methylated DNA;
(ii) an Fc portion of an antibody; and
(iii) a flexible peptide linker,
wherein the first polypeptide and second polypeptide each have the methyl-DNA-binding domain of the MBD2 protein fused to the Fc portion of an antibody through the flexible peptide linker; the fragment of the first polypeptide and the fragment of the second polypeptide each fused to the Fc portion of an antibody through the flexible peptide linker; or the polypeptide that is at least 70% homologous to the first polypeptide or fragment thereof and the polypeptide that is at least 70% homologous to the second polypeptide or the fragment thereof each fused to the Fc portion of an antibody through the flexible peptide linker; and
the Fc portion of the antibody fused to the first polypeptide is bonded to the Fc portion of the antibody fused to the second polypeptide; the Fc portion of the antibody fused to the fragment of the first polypeptide is bonded to the Fc portion of the antibody fused to the fragment of the second polypeptide; or the Fc portion of the antibody fused to the polypeptide that is at least 70% homologous to the first polypeptide or fragment thereof is bonded to the Fc portion of the antibody fused to the polypeptide that is at least 70% homologous to the second polypeptide or fragment thereof; and
(b) detecting the binding of the reagent to methylated DNA.

2. The method of claim 1, wherein step (b) comprises restriction enzyme digestion, bisulfate sequencing, pyrosequencing, Southern Blot, or PCR.

3. The method of claim 1, wherein step (b) comprises PCR.

4. The method of claim 1, further comprising step (c) analyzing the methylated DNA.

5. The method of claim 4, wherein analyzing the methylated DNA comprises sequencing.

6. The method of claim 1, wherein the container is coated directly or indirectly with the reagent.

7. The method of claim 1, wherein the sample is from a subject.

8. The method of claim 7, wherein the subject is suspected to have hypo- and/or hypermethylated gene loci.

9. The method of claim 8, wherein the hypo- and/or hypermethylated gene loci are indicative of a cancer, tumor or metastasis.

10. The method of claim 1, wherein less than about 10 ng of methylated DNA is detected in (b).

11. The method of claim 1, wherein less than about 5 ng of methylated DNA is detected in (b).

12. The method of claim 1, wherein the reagent comprises a polypeptide or fragment thereof that is at least 80% homologous with the first polypeptide or fragment thereof and is capable of binding methylated DNA and a polypeptide or fragment thereof that is at least 80% homologous to the second polypeptide or the fragment thereof and is capable of binding methylated DNA.

13. The method of claim 1, wherein the reagent comprises a polypeptide or fragment thereof that is at least 85% homologous with the first polypeptide or fragment thereof and is capable of binding methylated DNA and a polypeptide or fragment thereof that is at least 85% homologous to the second polypeptide or the fragment thereof and is capable of binding methylated DNA.

14. The method of claim 1, wherein the reagent comprises a polypeptide or fragment thereof that is at least 90% homologous with the first polypeptide or fragment thereof and is capable of binding methylated DNA and a polypeptide or fragment thereof that is at least 90% homologous to the second polypeptide or the fragment thereof and is capable of binding methylated DNA.

15. The method of claim 1, wherein the reagent comprises a polypeptide or fragment thereof that is at least 95% homologous with the first polypeptide or fragment thereof and is capable of binding methylated DNA and a polypeptide or fragment thereof that is at least 95% homologous to the second polypeptide or the fragment thereof and is capable of binding methylated DNA.

16. The method of claim 1, wherein MBD2 is human MBD2.

17. The method of claim 1, wherein MBD2 comprises amino acids 29 to 115 of SEQ ID NO:2.

18. The method of claim 1, wherein the flexible linker comprises amino acids 116 to 129 of SEQ ID NO:2.

19. The method of claim 1, wherein the binding of the reagent to methylated DNA is dependent on the degree of methylation.

20. The method of claim 1, wherein the binding of the reagent to methylated DNA is dependent on salt concentration.


SEQUENCE LISTING (23 sequences)

SEQ ID NO: 1 - DNA, 4598 bp, Homo sapiens
  CDS (851)..(1933)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccagtgaattttaacgttgcaggacaggatgtggtgcccgatgtgactagctctttgctgcaggccgtcctatcctctggttccgataagagacccagaactccggccccccaccgcccaccgccacccccatacatatgtggtacgcaagtaagagtgcctgcgcatgccccatgtgccccaccaagagttttgcatcccatacaagtccccaaagtggagaaccgaaccaattcttcgcgggcagaacaaaagcttctgcacacgtctccactcgaatttggagccggccggcgtgtgcaaaagaggtgaatcgaacgaaagacccgtgtgtaaagccgcgtttccaaaatgtataaaaccgagagcatctggccaatgtgcatcagttgtggtcagcagcaaaatcaagtgaatcatctcagtgcaactaaaggggggatccgatctcaatatgaagttatgcatattactggccgtcgtggcctttgttggcctctcgctcgggagatctccatggcccggggtacctactagcacggagagcgggaagaggatggattgcccggccctcccccccggatggaagaaggaggaagtgatccgaaaatctgggctaagtgctggcaagagcgatgtctactacttcagtccaagtggtaagaagttcagaagcaagcctcagttggcaaggtacctgggaaatactgttgatctcagcagttttgacttcagaactggaaagatgatgcctagtaaattacagaagaacaaacagagactgcgaaacgatcctctggcggccgcggatcccatcgaaggtcgtggtggtggtggtggtgatcccaaatcttgtgacaaacctcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagctagagggcccgcggttcgaaggtaagcctatccctaaccctctcctcggtctcgattctacgcgtaccggtcatcatcaccatcaccattgagtttaaacccgctgatcagcctcgactgtgccttctaaggcctgagctcgctgatcagcctcgatcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgt

SEQ ID NO: 2 - protein, 361 aa, Homo sapiens
MKLCILLAVVAFVGLSLGRSPWPGVPTSTESGKRMDCPALPPGWKKEEVIRKSGLSAGKSDVYYFSPSGKKFRSKPQLARYLGNTVDLSSFDFRTGKMMPSKLQKNKQRLRNDPLAAADPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 3 - protein, 361 aa, Homo sapiens; identical to SEQ ID NO: 2

SEQ ID NO: 4 - DNA, 28 bp, artificial sequence
  Primer, human MDB2 gene
agatgctagcacggagagcgggaagagg

SEQ ID NO: 5 - DNA, 35 bp, artificial sequence
  Primer, human MDB2 gene
atcacgcggccgccagaggatcgtttcgcagtctc

SEQ ID NO: 6 - DNA, 21 bp, artificial sequence
  Primer, human p15 gene
ggctcagcttcattaccctcc

SEQ ID NO: 7 - DNA, 19 bp, artificial sequence
  Primer, human p15 gene
aaagcccggagctaacgac

SEQ ID NO: 8 - DNA, 19 bp, artificial sequence
  Primer, human ESR1 gene
gactgcacttgctcccgtc

SEQ ID NO: 9 - DNA, 21 bp, artificial sequence
  Primer, human ESR1 gene
aagagcacagcccgaggttag

SEQ ID NO: 10 - DNA, 19 bp, artificial sequence
  Primer, human ICSBP gene
cggaattcctgggaaagcc

SEQ ID NO: 11 - DNA, 21 bp, artificial sequence
  Primer, human ICSBP gene
ttccgagaaatcactttcccg

SEQ ID NO: 12 - DNA, 21 bp, artificial sequence
  Primer, human METS gene
aattgcgtctgaagtctgcgg

SEQ ID NO: 13 - DNA, 20 bp, artificial sequence
  Primer, human METS gene
tcccacacaacagagaggcg

SEQ ID NO: 14 - DNA, 24 bp, artificial sequence
  Primer, human DP103 gene
gctgttagtccagttccaggttcc

SEQ ID NO: 15 - DNA, 22 bp, artificial sequence
  Primer, human DP103 gene
gtgcaaccacatttatctccgg

SEQ ID NO: 16 - DNA, 21 bp, artificial sequence
  Primer, human ICSBP gene
ggggtagttagtttttggttg

SEQ ID NO: 17 - DNA, 20 bp, artificial sequence
  Primer, human ICSBP gene
ataaataattccacccccac

SEQ ID NO: 18 - DNA, 21 bp, artificial sequence
  Primer, human ICSBP gene
ttgtggattttgattaatggg

SEQ ID NO: 19 - DNA, 22 bp, artificial sequence
  Primer, human ICSBP gene
ccrcccactatacctacctacc

SEQ ID NO: 20 - DNA, 18 bp, artificial sequence
  Primer, human ICSBP gene
cgtggtgtgcaaaggcag

SEQ ID NO: 21 - DNA, 25 bp, artificial sequence
  Primer, human ICSBP gene
ctgttatagaactgctgcagctctc

SEQ ID NO: 22 - DNA, 31 bp, artificial sequence
  Primer, human ACTB
tgacggggttcacccacactgtgcccatcta

SEQ ID NO: 23 - DNA, 30 bp, artificial sequence
  Primer, human ACTB
ctagaagcatttgtggtggacgatggaggg